(19) United States
(12) Patent Application Publication
(10) Pub. No.: US 2019/0041382 A1
(43) Pub. Date: Feb. 7, 2019

(54) SYSTEM AND METHOD FOR DETECTING THERAPEUTIC AGENTS TO MONITOR ADHERENCE TO A TREATMENT REGIMEN

(71) Applicant: UrSure, Inc., Boston, MA (US)

(72) Inventors: Helen Koenig, Boston, MA (US); Giffin Daughtridge, Boston, MA (US); Keith Kardos, Bethlehem, PA (US)

(21) Appl. No.: 16/078,960

(22) PCT Filed: Feb. 22, 2017
(86) PCT No.: PCT/US2017/018945

§ 371 Date: Aug. 22, 2018

Related U.S. Application Data
(60) Provisional application No. 62/298,231, filed on Feb. 22, 2016.

(51) Int. Cl. G01N 33/50; G01N 33/94

(52) U.S. Cl. CPC G01N 33/5038; G01N 33/94; G01N 33/5088

(57) ABSTRACT

The invention provides methods and systems for detecting a metabolite related to a NRTI in a biological sample, and use thereof in monitoring adherence to pre-exposure prophylaxis. The metabolite can be identified using proteomic methods, including but not limited to antibody based methods, such as a lateral flow immunoassay or lab based assays such as semi-quantitative LC-MS/MS.

### BACKGROUND OF THE INVENTION

Human Immunodeficiency Virus infects millions of individuals annually leading excessive morbidity and mortality as well as healthcare costs. Though it is a deadly and infectious disease, drug interventions have evolved to the point where the virus can be controlled in already infected patients. Two of the most widely used drugs for this purpose are the Nucleoside Reverse Transcriptase Inhibitors (NRTI) Tenofovir Disoproxil Fumarate (TDF) and Emtricitabine (FTC), which are often combined in the pill Truvada™.

In 2011, it was discovered that Truvada™ is also 99% effective at preventing HIV in HIV negative patients when taken daily as pre-exposure prophylaxis (PrEP). PrEP has been recommended in the U.S. by the CDC, and the World Health Organization globally, as a powerful tool for millions of individuals at risk for HIV. Daily adherence represents a challenge. Adherence to PrEP is critical for prevention of new infections, but patient self-report and pill counts are unreliable methods for monitoring adherence.

How to accurately identify suboptimal adherence, and develop strategic interventions to maintain adherence levels necessary for PrEP effectiveness in these populations, is the key gap in implementing this otherwise highly effective prevention therapy. Therapeutic drug monitoring has been useful for assessment of adherence in other fields, specifically adherence to psychiatric medications, treatment of substance abuse disorders, and for improved blood pressure control in patients with resistant hypertension. Furthermore, behavioral changes are maximized when feedback is made available close to the behavior that needs modification. Other means of measuring medication levels in patients receiving PrEP (plasma, dried blood spot, hair analysis) require invasive collection procedures that may not be acceptable to patients outside of clinical trials, are associated with delays in reporting that prevent implementation of timely effective interventions, and provide adherence information that may not be an adequate reflection of recent PrEP use.

To improve adherence, it is important that both the patient and provider be empowered to monitor levels of the drug in the body. Patients being monitored have demonstrated higher adherence to medications, and the information is also critical to informing targeted adherence counseling.

To date, monitoring methods for Tenofovir and Emtricitabine have proven invasive, painful, expensive, and do not provide real time data. As a result, they have not been amenable to patients nor particularly useful for providers. As a result, monitoring has not been a widely adopted method for improving drug adherence.

Adherence to PrEP correlates closely with efficacy. The iPrEX trial demonstrated that 2499 individuals at risk of contracting HIV who took TDF/FTC had a 44% reduction in HIV acquisition overall compared to those who took placebo, vs a 99% reduction in those who took PrEP daily (Grant 2010; Prejean 2011). An intracellular tenofovir-diphosphate concentration of 16 fmol per million peripheral blood mononuclear cells (PBMCs) was associated with a 90% reduction in HIV acquisition relative to the placebo arm (Anderson 2012). Directly observed dosing in the STRAND study yielded intracellular tenofovir concentrations that corresponded with HIV-1 risk reduction of 76% for 2 doses per week, 96% for 4 doses per week, 99% for 7 doses per week (Prejean 2011).The importance of adherence was also demonstrated in the TDF2 trial of heterosexual men and women in which efficacy of TDF/FTC was 63% overall and 78% in consistent users (Thigpen 2012), and in the Partners PrEP trial of in which efficacy was 73% overall and 90% in consistent users (Baeten 2012). In contrast, TDF/FTC was found to be ineffective in preventing HIV infection in the Fem-PrEP (Van 2012) and VOICE trials, in which adherence was demonstrated to be extremely poor.

Current measurements of adherence to PrEP are inadequate. Self-reported adherence and pharmacy refill data alone do not correlate well with actual adherence in PREP trials (Amico, K. R. 2014). In individuals at risk of contracting HIV in an urban setting, rates of detectable plasma tenofovir levels ranged from 63.2% at week 4 to 20% at week 24 after the initiation of PrEP despite high levels of self-reported medication adherence (Hosek 2012). The same was true of trials looking at women such as the Fem-PrEP trial: less than 40% of a representative sample of study subjects had detectable drug in plasma, despite 95% of women reporting that they “always” or “usually” used the product, and pill counts suggesting that study drug was taken on 88% of days (Amico 2013; Van 2012). Thus, there is a need in the art for systems and devices that allow for accurate, easy and consistent monitoring of these drugs' levels in the patients' body.

Point of care testing (POCT) provides clinicians with rapid results for many commonly ordered tests, including blood glucose, urine testing for infection, urine pregnancy testing, fecal occult blood, and rapid HIV testing. These tests are typically done within the clinic setting in order to provide information during the course of a patient encounter that can help inform decisions around patient care and improve the relationship between clinicians and patients by enhancing communication and shared decision-making (Jones, C. H. 2013). POCT are used in several environments: hospital bedside, ambulatory care settings (clinics or physician offices), alternate care (skilled nursing facilities), and home settings. Before a POC kit or device can be legally marketed and sold, its labeling must be approved by the United. States Food and Drug Administration (FDA). POCT is accomplished through the use of transportable, portable, and handheld instruments (e.g., blood glucose meter) and test kits (e.g., CRP, HbAlc, homocysteine, HIV salivary assay, etc.). Small, mobile bench top analyzers or fixed equipment can also be used when handheld devices are not available. The number of POC tests currently available is growing exponentially from fewer than 10 tests available in 1995 to approximately 110 tests available today. The use of POCT has transformed many aspects of clinical medicine, like monitoring glycemia in patients with diabetes mellitus, monitoring the use and abuse of illegal substances, monitoring of oral anticoagulation and the diagnosis of pregnancy, to name a few. POCT applications under consideration or development include monitoring HIV disease in the developing world, monitoring lactate, CD4, HIV mRNA viral load, and drug resistant tuberculosis strains in HIV-positive patients diagnosed with AIDS (Stevens, 2010). In the field of HIV in particular, POCT to diagnose HIV has completely transformed the ability to link patients to care quickly, especially in resource limited settings where health infrastructure may be poor and timely access to medical care is difficult (Arora, D. R. 2013). Furthermore, data suggest that individuals who are aware of their HIV status are more likely to adopt risk reduction behaviors than those who are not.

Therapeutic drug monitoring (TDM) has been effectively used to help physicians monitor and maintain drug levels within the therapeutic window in other clinical settings. TDM been found to be useful to identify inadequate adherence as a cause of poor treatment response in a variety of fields (Brunen 2011; Brunen 2011; Hiemke 2008; Brinker 2014). In particular, TDM has been found to be a suitable tool for assessment of adherence to psychiatric medications in psychiatric outpatients with co-morbid substance abuse disorders (Brunen 2011), in treatment of substance abuse disorders for certain reference drugs including bupropion, buprenorphine, disulfiram, methadone, and naltrexone (Brumen 2011), and for improved blood pressure control in patients who have been diagnosed with resistant hypertension (Brinker 2014). Additional limitations to TDM mentioned in the literature include “white coat compliance” (improved adherence preceding a clinic visit) that may limit the ability to rely completely on results of TDM (Podsadecki 2008), and the concern that TDM may not be appropriate for all clinical settings in its current form. Therefore, there is a need in the art for POCT for TDM that can be used both in the clinical setting and outside of clinical settings, which can function as a powerful tool in conjunction with good patient communication.

The current invention addresses these needs by providing a POCT for detecting a therapeutic agent in order to monitor adherence to a treatment regimen.

### SUMMARY OF THE INVENTION

In one aspect, the invention provides a system for detecting a metabolite in a biological sample from a subject. In one embodiment, the system is useful for detecting a therapeutic agent in order to monitor adherence to a treatment regimen.

In one embodiment, the biological sample is at least one sample selected from the group comprising a urine sample, a saliva sample, a mucous sample, a whole blood sample, a blood plasma sample and a milk sample obtained from the subject.

In one embodiment, the metabolite is from a therapeutic agent. In one embodiment, the metabolite is from a prophylactic agent. In one embodiment, the metabolite is from an NRTI. In one embodiment, the NRTI is at least one of the group comprising Tenofovir Disoproxil Fumarate (TDF), Emtricitabine (FTC), and Tenofovir Alafenamide (TAF), or derivatives thereof or combinations thereof.

In one embodiment, the invention relates to monitoring the presence of a metabolite in an individual. In one embodiment, the invention relates to monitoring adherence to a pre-exposure prophylaxis in an individual, wherein a concentration of about 1,000 ng/mL or more of a metabolite from a NRTI in a urine sample from a patient is identified as adherent.

In one embodiment, the invention relates to counseling an individual at risk of contracting HIV, wherein concentration of about 999 ng/mL or less of a metabolite from a NRTI in a urine sample from a patient is identified as risk of contracting HIV.

The invention also relates to a method of identifying a metabolite in a biological sample from a subject. In one embodiment, the biological sample is at least one sample selected from the group comprising a urine sample, a saliva sample, a mucous sample, a whole blood sample, a blood plasma sample and a milk sample obtained from the subject.

In one embodiment, the invention relates to a method of identifying a metabolite in a sample comprising applying a biological sample obtained from the subject to a system, wherein the system comprises at least one method for detection of the metabolite. In one embodiment, the system comprises at least one molecule that specifically binds at least one metabolite. In one embodiment, the system comprises an immunoassay for detection of at least one metabolite. In one embodiment, the system comprises a lab based method. In one embodiment, the lab based method is LC-MS/MS.

In one embodiment, the invention relates to a method of identifying a metabolite from a NRTI in a sample. In one embodiment, the NRTI is selected from the group comprising TDF, FTC, TAF, or derivative thereof and any combination thereof. In one embodiment, risk of contracting HIV is diagnosed when a NRTI is or is not detected.

In one aspect, the invention relates to a kit comprising a system for detecting a metabolite in a biological sample from a subject. In one embodiment, the biological sample is at least one sample selected from the group comprising in of a urine sample, a saliva sample, a mucous sample, a whole blood sample, a blood plasma sample and a milk sample obtained from the subject.

In one embodiment, the invention relates to a kit comprising a system for detecting a metabolite in a biological sample from a subject, wherein the metabolite is from a therapeutic agent. In one embodiment, a metabolite for detection by a kit of the invention is from a NRTI. In one embodiment, the NRTI is at least one of the group comprising TDF, TAF, and FTC, or derivatives thereof or combinations thereof. In one embodiment, the system for detecting a metabolite comprises an immunoassay. In one embodiment, the system for detecting a metabolite comprises a point of care device.

In one aspect, the invention relates to the use of a kit comprising a system for detecting a metabolite in a biological sample from a subject for monitoring NRTI in an individual. In one embodiment, use of the kit includes monitoring adherence to a treatment regimen in an individual. In one embodiment, use of the kit includes monitoring adherence to a prophylactic regimen in an individual. In one embodiment, use of the kit includes monitoring adherence to a pre-exposure prophylaxis in an individual. In one embodiment, use of the kit includes counseling an individual. In one embodiment, the individual is at risk of contracting HIV.

Another aspect of the invention relates to a method for detecting a metabolite in a patient, the method comprising:

a) administering an effective amount of a Nucleoside Reverse Transcriptase Inhibitor (NRTI) to the patient;

b) obtaining a biological sample from the patient;

c) detecting whether the metabolite is present in the sample; and

d) determining adherence to a treatment or prophylactic regimen in the patient.

In one embodiment, the metabolite is tenofovir (TFV).

In one embodiment, the NRTI is selected from the group consisting of Tenofovir Disoproxil Fumarate (TDF), Emtricitabine (FTC), and Tenofovir Alafenamide (TAF), or derivatives thereof or combinations thereof.

In one embodiment, the NRTI is TAF.

In one embodiment, the NRTI is TDF.

In one embodiment, the NRTI is FTC.

In one embodiment, the NRTI is a combination of TDF/FTC.

In one embodiment, the NRTI is a combination of TAF/FTC,

In one embodiment, in step c), the metabolite is detected by immunoassay or spectrometry.

In one embodiment, the spectrometry is selected from the group consisting of matrix-assisted laser desorption/ionization time-of-flight (MALDI-TOF) mass spectroscopy (MS), MALDI-TOF post-source-decay (PSD), MALDI-TOF/TOF, surface-enhanced laser desorption/ionization time-of-flight (SELDI-TOF) MS, tandem MS, electrospray ionization MS (ESI-MS), ESI-MS/MS, ESI-MS/(MS)n, ESI 3D ion trap MS, ESI linear (2D) MS, ESI triple quadrupole MS, ESI quadrupole orthogonal TOF (Q-TOF), ESI Fourier transform MS, desorption/ionization on silicon (DIOS), secondary ion MS (SIMS), atmospheric pressure chemical ionization MS (APCI-MS), APCI-MS/MS, atmospheric pressure photoionization MS (APPI-MS); APPI-MS/MS, APCI-(MS)ⁿ, liquid chromatography MS(LC-MS), semi-quantitative liquid chromatography-tandem (LC-MS/MS), gas chromatography-MS (GC-MS), high performance liquid chromatography-MS (HPLC-MS), capillary electrophoresis-MS, and nuclear magnetic resonance (NMR) spectrometry.

In one embodiment, the spectrometry is LC-MS/MS.

In one embodiment, the biological sample is selected from the group comprising urine, saliva, mucous, whole blood, and blood plasma.

In one embodiment, the biological sample is urine.

In one embodiment, the patient is negative for human immunodeficiency virus (HIV), positive for HIV, or at risk for HIV.

In one embodiment, the patient is negative for HIV, and said patient is at high risk for exposure to HIV.

In one embodiment, the metabolite has a concentration of about 0 ng/ml to about 10 ng/mL.

In one embodiment, the metabolite has a concentration of about 10 ng/ml to about 10,000 ng/mL.

In one embodiment, the metabolite has a concentration of about 10 ng/ml to about 1,000 ng/mL.

In one embodiment, the metabolite in step c) is contacted with a reagent to detect the metabolite.

In one embodiment, the reagent is an antibody.

In one embodiment, the antibody is a polyclonal antibody.

In one embodiment, the antibody is a monoclonal antibody.

In one embodiment, the prophylactic regimen is a pre-exposure prophylaxis (PrEP).

In one embodiment, the PrEP is for 28 days.

In one embodiment, the prophylactic region is a post exposure prophylaxis (PEP).

In one embodiment, the PEP is administered within 72 hours of a high risk exposure and continued for at least 28 days.

In one embodiment, in step d), recent adherence to the treatment or prophylactic regimen in the patient is determined.

In one embodiment, recent adherence is defined by a dose of NRTI within 48 hours.

In one embodiment, recent adherence is defined by a metabolite concentration of 1000 ng/mL or more.

In one embodiment, recent adherence to the prophylactic regimen in the patient identifies the patient as at little to no risk of contracting HIV.

In one embodiment, in step d), low adherence to the treatment or prophylactic regimen in the patient is determined.

In one embodiment, low adherence is defined by a dose of NRTI within 1 week.

In one embodiment, low adherence is defined by a metabolite concentration of 10 ng/mL, to 999 ng/mL.

In one embodiment, low adherence to the prophylactic regimen in the patient identifies the patient as at risk of contracting HIV.

In one embodiment, in step d), non-adherence to the treatment or prophylactic regimen in the patient is determined.

In one embodiment, non-adherence is defined by a last dose of NRTI greater than 1 week.

In one embodiment, non-adherence is defined by a metabolite concentration of 10 ng/mL or less.

In one embodiment, non-adherence to the prophylactic regimen in the patient identifies the patient as at high risk of contracting HIV.

Another aspect of the invention relates to a diagnostic system for carrying out any of the aforementioned methods.

Another aspect of the invention relates to an immunoassay for carrying out any of the aforementioned methods.

In one embodiment, the immunoassay is a competitive assay.

Another aspect of the invention relates to a device for performing an assay to detect a metabolite in a fluid sample of a patient, wherein the patient is prescribed or administered an NRTI, comprising:

(a) a sample pad for contacting the fluid sample;

(b) a conjugated label pad, the conjugated label pad having a first reagent conjugated to a detectable label, a portion of the conjugated label pad and a portion of the sample pad forming a first interface;

(c) an assay comprising a membrane, a portion of the membrane and a portion of the conjugated label pad forming a second interface; and

(d) at least one second reagent bound to the membrane to form a test line, the first interface allowing fluid to flow from the sample pad to the conjugated label pad and contact the detectable label, the second interface allowing fluid to flow from the conjugated label pad to the membrane and to contact the at least one membrane-bound second reagent to form to a second reagent-first reagent complex, and cause the detectable label to form a detectable signal at the test line,

wherein the presence of a detectable signal indicates non-adherence to a treatment or prophylactic regimen in the patient, and wherein the absence of a detectable signal indicates adherence to a treatment or prophylactic regimen in the patient.

In one embodiment, the detectable signal is modulated to provide that the presence of a detectable signal indicates adherence to a treatment or prophylactic regimen in the patient.

In one embodiment, the assay is a lateral flow assay.

In one embodiment, the assay is a lateral flow immunoassay.

In one embodiment, the first reagent is an antibody conjugated to a detectable label.

In one embodiment, the first reagent is a conjugated derivative of the metabolite.

In one embodiment, the second reagent is a conjugated derivative of the metabolite.

In one embodiment, the second reagent is an antibody to the metabolite.

In one embodiment, the first reagent is an antibody conjugated to a detectable label and the second reagent is a conjugated derivative of the metabolite.

In one embodiment, the first reagent is a conjugated derivative of the metabolite and the second reagent is an antibody to the metabolite.

In one embodiment, the device further comprises an absorbent pad downstream of the membrane.

In one embodiment, the membrane is nitrocellulose.

In one embodiment, the device is provided in a housing.

In one embodiment, the housing further comprises an opening for reading the detectable signal.

In one embodiment, the first reagent is an antibody specific for the metabolite.

In one embodiment, the antibody is a polyclonal antibody.

In one embodiment, the antibody is a monoclonal antibody.

In one embodiment, the metabolite is TFV.

In one embodiment, the metabolite is a TFV derivative.

In one embodiment, the membrane further comprises a third reagent bound to the membrane downstream or upstream of the test line to form a control line.

In one embodiment, the third reagent binds to the first reagent to cause a detectable signal at the control line, wherein the presence of the detectable signal at the control line indicates proper performance of the lateral-flow assay.

In one embodiment, the device is a point of care test.

In one embodiment, the device is a cartridge.

In one embodiment, the fluid sample is urine.

In one embodiment, the prophylactic regimen is a PrEP to NRTI.

In one embodiment, the NRTI is selected from the group consisting of TDF, FTC, and TAF, or derivatives thereof or combinations thereof.

In one embodiment, the NRTI is TAF.

In one embodiment, the NRTI is TDF.

In one embodiment, the NRTI is FTC.

In one embodiment, the NRTI is a combination of TDF/FTC.

In one embodiment, the NRTI is a combination of TAF/FTC.

Another aspect of the invention relates to a kit, comprising:

(a) a sample collection receptacle for receiving a biological sample; and

(b) the device of claim 42 for assaying the biological sample;

In one embodiment, the kit further comprises instructions for use.

In one embodiment, the kit further comprising a hand held device.

In one embodiment, the hand held device is a reader.

In one embodiment, the reader is adapted to receive the device of claim 28.

In one embodiment, the reader is a reflectance reader.

In one embodiment, the sample is urine.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a chart of the Tenofovir (TFV) calibration curve in human urine over a concentration range of 10 to 1000 ng/mL. A known concentration of TFV was injected into a human urine sample (x axis) and measured using LC-MS/MS (y axis). [TFV]=tenofovir concentration (ng/mL).

FIG. 2 depicts experimental results showing the relationship between urine and plasma concentration in well-controlled individuals. All subjects with TFV in plasma were also detected as having TFV in urine. Concentrations in plasma (P) and urine (U) are shown for each subject.

FIG. 3 depicts a graph detailing the clearance or rate of decay of TFV in plasma and urine over seven days following TDF/FTC administration.

FIG. 4 depicts the results of a study using a urine assay for TDF/FTC in a cohort of patients on PrEP.

FIG. 5 depicts a representative MS/MS chromatogram of tenofovir (100 ng/mL in human plasma) and an internal standard.

### DETAILED DESCRIPTION

The present invention relates to systems and methods for conveniently detecting the presence or absence of a therapeutic agent in a sample as well as determining variable levels of the therapeutic agent. In one embodiment, the therapeutic agent is a prophylactic agent.

In one embodiment, the invention provides a method for conveniently detecting the presence or absence of a therapeutic agent in a patient sample. The patient sample can be one or more of a urine sample, a saliva sample, a blood sample and a plasma sample. In one embodiment, the sample is from a patient who has been prescribed a therapeutic agent as part of a treatment regimen.

In one embodiment, the sample is from a patient diagnosed with HIV. In sonic instances, the sample is from a patient at risk of HIV infection. In one embodiment, the sample is from a patient who has been prescribed a NRTI as part of a treatment regimen. In one embodiment, the treatment regimen is prophylactic. In some instances, the sample is from a patient who is taking Truvada™ for treatment of HIV. In some instances, the sample is from a patient who is taking Truvada™ as a PrEP.

In one embodiment the sample is from a patient who is taking an NRTI. In one embodiment, the NRTI is TDF. In one embodiment, the NRTI is FTC. In one embodiment, the NRTI is both TDF and FTC. In one embodiment, the NRTI is Truvada™. In one embodiment, the NRTI is TAF. In one embodiment, the NRTI is both TAF and FTC.

In one embodiment, the invention relates to a device that can be used for detecting NRTI in a specimen. In one embodiment, the invention provides a system for detection of NRTI in a form of a POCT. In one embodiment, the invention provides a system for detection of NRTI in a form of a hand held device. In one embodiment, a hand held device may interact with a POCT, such as a test strip. In one embodiment, a hand-help device may interface with a computer software, an application (app), or a web-based evaluation tool. In one embodiment, a computer software, app, or web-based evaluation tool can provide results to a physician for monitoring adherence to a prophylactic treatment regimen. In one embodiment, a handheld device interfacing with a computer software is useful for self-monitoring by an individual.

In another embodiment, the method of the invention may comprise any method known in the art to effectively detect a NRTI in a sample. Suitable methods include, but are not limited to, immunoassays, enzyme assays, mass spectrometry, biosensors, and chromatography. Thus, the method of the invention includes the use of any type of instrumentality to detect a NRTI.

The invention relates to the discovery that one or more NRTIs is present in the urine of a patient who has taken an NRTI. Occurrence of the NRTI in a patient's urine is an indicator that the patient has taken a prescribed NRTI. The amount of the NRTI decreases with time in the absence of subsequent doses of the NRTI. Thus, the invention can be used to assess the level of adherence to a prescribed treatment plan for a patient prescribed an NRTI. In one embodiment, the invention can be used to assess the NRTI level of an individual who has previously taken an NR H before an episode wherein the individual is at risk of contracting HIV. Accordingly, the method of the invention provides a new and convenient platform for monitoring adherence and response to a particular treatment.

In some instances, the invention may take the form of a user-friendly point-of-use or point-of-care platform, for example a lateral flow device, having a sample application region and a readable detection region to indicate the presence or absence of the NRTI or variable levels of the NRTI. In one embodiment, the readable detection region includes a test line and a control line, wherein the test line detects the NRTI, and the control line detects the presence or absence of a marker present in the fluid being tested. Preferably, the fluid being tested is urine and the marker includes, but is not limited to IgG, IgD or IgA.

In one embodiment, a comparison of the control line to the test line yields the test result. In some instances, a valid result occurs when the control line is detected at a higher intensity level than the test line. For example, a valid result occurs when the control line is darker than the test line. That is, the control line represents an internal control for the diagnostic system of the invention for verifying that the sample being evaluated is urine.

In one embodiment, the control line is a reference line that insures that the test has been run correctly. The control line is also used as a reference when the reader determines if the result is positive or negative. For example, the system of the invention is useful for detecting an NRTI in a sample when the control line is detected at a higher intensity than the test line. In some instances, if the test line is darker than the control line, then the test is said to have an invalid result. If the test line is lighter than the control line then the test is said to have a valid result.

In one embodiment, the system of the invention detects a NRTI by way of a lateral flow immunoassay that utilizes strips of cellulose membrane onto which antibodies and other reagents are applied. For example, the test sample moves along the strip due to capillary action and reacts with the reagents at different points along the strip. The end result is the appearance or absence of a detectable line or spot.

In one embodiment, the lateral flow device can be in the form of a cartridge that can be read by a machine. Preferably, the machine is automated.

In one embodiment, the NRTI of the invention can be detected in a system that takes the form of a laboratory test, for example a type of numbered well plate (e.g., 96 well plate).

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in sonic instances ±10%, or in some instances ±5%, or in some instances ±1%, or in some instances ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “affinity moiety” refers to a binding molecule, such as an antibody, aptamer, peptide or nucleic acid, that specifically binds to a particular target molecule, such as an analyte, metabolite or other targeted molecule to be detected in a testing sample.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y., Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “applicator,” as the term is used herein, is meant any device including, but not limited to, a hypodermic syringe, a pipette, an iontophoresis device, a patch, and the like, for administering the compositions of the invention to a subject.

As used herein, “metabolite” or “marker” in the context of the present invention encompasses, without limitation, analytes and metabolites, together with degradation products, protein-ligand complexes, elements, related metabolites, and other analytes or sample-derived measures. Marker also includes any calculated indices created mathematically or combinations of any one or more of the foregoing measurements, including temporal trends and differences.

The terms “metabolite related to NRTI” and “NRTI” are used interchangeably herein. Therefore it should be understood that a reference to “NRTI” should be read as relating to any metabolite specifically associated with a “NRTI”. As a non-limiting example, Tenofovir (TFV) is an active metabolite related to the NRTI Tenofovir Disoproxil Fumarate (TDF) and Tenofovir Alafenamide (TAF).

As used herein, a “biosensor” is an analytical device for the detection of an analyte in a sample. Biosensors can comprise a recognition element, which can recognize or capture a specific analyte, and a transducer, which transmits the presence or absence of an analyte into a detectable signal.

As used herein, the term “data” in relation to one or more metabolites, or the term “metabolite data” generally refers to data reflective of the absolute and/or relative abundance (level) of a product of a metabolite in a sample. As used herein, the term “dataset” in relation to one or more metabolites refers to a set of data representing levels of each of one or more metabolite products of a panel of metabolites in a reference population of subjects. A dataset can be used to generate a formula/classifier of the invention. According to one embodiment, the dataset need not comprise data for each metabolite product of the panel for each individual of the reference population. For example, the “dataset” when used in the context of a dataset to be applied to a formula can refer to data representing levels of each metabolite for each individual in one or more populations, but as would be understood can also refer to data representing levels of each metabolite for 99%, 95%, 90%, 85%, 80%, 75%, 70% or less of the individuals in each of said one or more populations and can still be useful for purposes of applying to a formula.

The term “control” or “reference standard” describes a material comprising none, or a normal, low, or high level of one of more of the marker (or metabolite) of the invention, such that the control or reference standard may serve as a comparator against which a sample can be compared.

As used herein, the term “detection reagent” refers to an agent comprising an affinity moiety that specifically binds to an analyte, metabolite or other targeted molecule to be detected in a sample. Detection reagents may include, for example, a detectable moiety, such as a radioisotope, a fluorescent label, a magnetic label, and enzyme, or a chemical moiety such as biotin or digoxigenin. The detectable moiety can be detected directly, or indirectly, by the use of a labeled specific binding partner of the detectable moiety. Alternatively, the specific binding partner of the detectable moiety can be coupled to an enzymatic system that produces a detectable product.

As used herein, a “detector molecule” is a molecule that may be used to detect a compound of interest. Non-limiting examples of a detector molecule are molecules that bind specifically to a compound of interest, such as, but not limited to, an antibody, a cognate receptor, and a small molecule.

By the phrase “determining the level of marker (or metabolite)) concentration” is meant an assessment of the amount of a marker in a sample using technology available to the skilled artisan to detect a sufficient portion of any marker product.

A “disease” is a state of health of an animal wherein the animal cannot maintain homeostasis, and wherein if the disease is not ameliorated then the animal's health continues to deteriorate.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

As used herein, an “instructional material” includes a publication, a recording, a diagram, or any other medium of expression which can be used to communicate the usefulness of a component of the invention in a kit for detecting metabolites disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more metabolites means the absolute or relative amount or concentration of the metabolite in the sample.

A “marker,” as the term is used herein, refers to a molecule that can be detected. Therefore, a marker according to the present invention includes, but is not limited to, a nucleic acid, a polypeptide, a carbohydrate, a lipid, an inorganic molecule, an organic molecule, an analyte, a metabolite or a radiolabel, each of which may vary widely in size and properties. A “marker” can be detected using any means known in the art or by a previously unknown means that only becomes apparent upon consideration of the marker by the skilled artisan. A marker may be detected using a direct means, or by a method including multiple steps of intermediate processing and/or detection. The term “tag” is also used interchangeably with the term “marker,” but the term “tag” may also be used, in certain aspects, to include markers that are associated with one or more other molecules.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

As used herein, the “monitoring adherence” refers to determining compliance of a patient with a prescribed course of treatment. Adherence encompasses compliance with aspects including dosage amounts and frequencies of a prescribed course of treatment.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Polypeptide,” as used herein refers to a polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” or “peptide” as used herein are intended to encompass any amino acid sequence and include modified sequences such as glycoproteins. The term “polypeptide” or “protein” or “peptide” is specifically intended to cover naturally occurring proteins, as well as those which are recombinantly or synthetically produced. It should be noted that the term “polypeptide” or “protein” includes naturally occurring modified forms of the proteins, such as glycosylated forms.

As used herein, the term “providing a prognosis” refers to providing a prediction of the probable course and outcome of a disease, disorder or condition, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a metabolite means a level of the metabolite that is indicative of a therapeutic level of the drug.

The term “risk” according to the invention, comprises finding a particular patient who is not currently diagnosed with HIV may become exposed to bodily fluid from an individual currently diagnosed with HIV or otherwise become exposed to HIV.

“Sample”, “specimen” or “biological sample” as used herein means a biological material isolated from an individual. The biological sample may contain any biological material suitable for detecting the desired metabolites, and may comprise cellular and/or non-cellular material obtained from the individual.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The “therapeutic concentration” or “therapeutic level” is the concentration of a substance at which therapeutic benefits are gained. For NRTIs of the invention, the therapeutic concentration is about 1,000 ng/mL, or more.

The term “treatment regimen” or “medical regimen” as used herein relates to at least the frequency and dosage of any pharmaceutical agent being taken by an individual for treatment or prevention of a disease or condition.

### Description

The present invention relates to systems and methods for conveniently monitoring the presence or absence of NRTI in a sample. Preferably, the sample is urine. Occurrence of the NRTI in a patient's urine is an indicator that the patient has taken a prescribed NRTI. In one embodiment, the invention can be used to assess the level of adherence to a prescribed treatment plan for a patient prescribed an NRTI. In one embodiment, the invention can be used to assess the NRTI level of an individual who has previously taken an NRTI before an episode wherein the individual is at risk of contracting HIV. Accordingly, the method of the invention provides a new and convenient platform for monitoring adherence and response to a particular treatment.

The invention provides methods and systems for detecting a NRTI in urine wherein the system also includes a control in order to ensure that the test sample is indeed urine. The NRTI and the control for urine may be identified by any suitable assay. A suitable assay may include one or more of an enzyme assay, an immunoassay, mass spectrometry, chromatography, electrophoresis, a biosensor, an antibody microarray, or any combination thereof. If an immunoassay is used it may be an enzyme-linked immunosorbant immunoassay (ELISA), a sandwich assay, a competitive assay, a radioimmunoassay (RIA), a lateral flow immunoassay, a Western Blot, an immunoassay using a biosensor, an immunoprecipitation assay, an agglutination assay, a turbidity assay or a nephelometric assay. A preferred method is an immunoassay that utilizes a rapid immunoassay platform such as lateral flow.

Accordingly, the invention includes any platform for detecting a NRTI in a biological sample such as urine. In one embodiment, the system provides a convenient point-of-care device which can quickly detect the presence or absence of a NRTI in an at home or clinical setting. One non-limiting example of a point of care device is a lateral flow immunoassay. Lateral flow immunoassay utilizes strips of a membrane, preferably a cellulose membrane such as nitrocellulose, as the solid support for the immunoassay, onto which lines of reagent (e.g. antibody or antigen specific for the target analyte) can be applied. Multiple analytes can be assayed by spatially separating the location of the application areas of the reagents. Additional reagent pads can be used below the test line(s) for other critical reagents and sample conditioning materials. When sample is added to the test device, the solution will flow across the pads below the test lines and rehydrate the sample conditioning compound and the critical reagents for the assay and then pass across the specific test line and deposit a detection label which can be a visual indication (colloidal gold, colored latex or other labels known to those skilled in the art) or a label that requires an instrument to measure the signal (fluorescence, chemiluminesence). An additional material can be added above the test line to absorb fluid that passes by the test lines.

The end result is the appearance or absence of a colored line or spot, which can be compared to a control line. In some instances, the control line is useful for the detection of a marker of urine in order to ensure that the sample tested is indeed urine. Preferably, the marker of urine is present at a concentration significantly different in urine compared to the amount in other common matrices (i.e. blood) so as to validate that the sample tested is urine.

In one embodiment, the system may include a base or support layer and an absorbent matrix comprising at least one absorbent layer through which a liquid sample can flow along a flow path by force or by capillary action. The base layer may also be absorbent and be in fluid communication with the absorbent matrix, such that the flow path of liquid sample passes through both the absorbent matrix and the base layer. The flow path includes at least two regions, where the first region is a sample application region, and the second region is a detection region.

In one embodiment, immunoassays can be formatted in a sandwich format where two antibodies or binding partners specific for a molecule can be utilized to anchor and detect the analyte of interest. Smaller molecules can be detected using a competitive format where only one antibody or binding partner is utilized to detect the drug of interest. The assays can be formatted in a method that provides a positive read, in which a line appears when drug is present, or a negative read, in which the line disappears when the drug is present.

One embodiment of the invention involves the production of antibodies or binding partners with high specificity to the drug or drug metabolite of interest for utilization in the immunoassay. The antibody should have high specificity to the target drug or drug metabolite to permit the design of an immunoassay that allows monitoring of compliance of drug dosing. The production of the antibody will require the synthesis of a derivative that can be utilized to immunize animals. The derivative will be designed in a manner to maximize the recognition of the target molecule with minimal cross reactivity to other substances that may be present in the sample. The derivative is linked to a carrier protein to enhance the immune recognition and allow the production of antibodies. The antibodies can be polyclonal or more preferably monoclonal antibodies. The design and production of antibodies is well known to those skilled in the art.

In one embodiment of the invention, the test device is a competitive immunoassay utilizing a lateral flow format with a negative read out that measures a single drug substance. The lateral flow strip has a sample pad that contains the buffering and sample treatment materials. The sample pad is in contact with a conjugate pad that contains a label linked to a derivative of the drug substance. The conjugate pad is in contact with a solid support, such as nitrocellulose, that has had an antibody striped onto it and also has a control line that has an antibody or binding partner that will bind the conjugate in both the presence and absence of the target drug. The test device may have an absorbent pad downstream from the test zones to facilitate flow through the device. The device may optionally have a device housing to contain the strip and create an opening for the addition of sample to the device. The presence of a line in the test zone and the control zone would indicate that the subject had not been routinely taking the target drug and the absence of a line would indicate that they had been taking the drug.

In one embodiment of the invention, the test device is a competitive immunoassay utilizing a lateral flow format with a negative read out that measures a single drug substance. The lateral flow strip has a sample pad that contains the buffering and sample treatment materials. The sample pad is in contact with a conjugate pad that contains a label linked to an antibody made to the drug substance. The conjugate pad is in contact with a solid support, such as nitrocellulose, that has had a derivative of the target drug striped onto it and also has a control line that has an antibody or binding partner that will bind the conjugate in both the presence and absence of the target drug. The test device may have an absorbent pad downstream from the test zones to facilitate flow through the device. The device may optionally have a device housing to contain the strip and create an opening for the addition of sample to the device. The presence of a line in the test zone and the control zone would indicate that the subject had not been routinely taking the target drug and the absence of a line would indicate that they had been taking the drug.

In one embodiment of the invention, the test device is a competitive immunoassay utilizing a lateral flow format with a positive read out that measures a single drug substance. The lateral flow strip has a sample pad that contains the buffering and sample treatment materials. The sample pad is in contact with a conjugate pad that contains a label that is linked to an antibody made to the drug substance. The conjugate pad is in contact with a solid support, such as nitrocellulose, that has had a derivative of the target drug striped onto it at a position that is not visible to the user and a binding partner for the conjugate not related to the drug at the test line (ex Avidin/Biotin). The solid support also has a control line that has an antibody or binding partner that will bind a secondary conjugate to indicate that the device has been run. The test device may have an absorbent pad downstream from the test zones to facilitate flow through the device. The device may optionally have a device housing to contain the strip and create an opening for the addition of sample to the device. The presence of a line in the test zone and the control zone would indicate that the subject had been routinely taking the target drug and the absence of a line would indicate that they had not been taking the drug.

In one embodiment of the invention, the test device is a competitive immunoassay utilizing a lateral flow format with a negative read out that measures a combination of drug substances. The lateral flow strip has a sample pad that contains the buffering and sample treatment materials. The sample pad is in contact with a conjugate pad that contains a label linked to 2 or more derivatives of drug substances. The conjugate pad is in contact with a solid support, such as nitrocellulose, that has had an antibodies striped onto it at 2 or more test positions and also has a control line that has an antibody or binding partner that will bind the conjugate in both the presence and absence of the target drug. The test device may have an absorbent pad downstream from the test zones to facilitate flow through the device. The device may optionally have a device housing to contain the strip and create an opening for the addition of sample to the device. In this embodiment, the pattern of reactivity of the 2 or more drugs could indicate the adherence to the recommended dosing for the drugs. In one potential outcome, a lateral flow test readout of two positive test lines or spots could indicate that the individual providing the sample was taking a NRTI according to the prescribed dosage schedule, whereas a lateral flow test readout of one positive test line or spot could indicate that the individual providing the sample was taking a NRTI but not according to the prescribed dosage schedule, and a lateral flow test readout of zero positive test lines or spots could indicate that the individual providing the sample was not taking a NRTI.

In one embodiment, the NRTI of the invention can be detected in a system that takes the form of a laboratory test, for example a type of numbered well plate (e.g., well plate). In one embodiment, the lateral flow device can be in the form of a cartridge that can be read by a machine. Preferably, the machine is automated.

In one embodiment, the system of the invention includes (i) a POCT and (ii) a digital device. In one embodiment, a digital device interacts with a POCT. In one embodiment, a digital device analyzes the results from a POCT. In one embodiment, a digital device records the results from a POCT. In one embodiment, a digital device reports the results from a POCT. In one embodiment, a digital device analyzes, records and/or reports the results from multiple POCT.

The invention disclosed is not limited to the platform chosen to measure the NRTI concentrations. Rapid tests are well known and can be formatted in a lateral flow, flow through, capillary, biosensor and a number of other formats.

### Health Profile

In one embodiment, the present invention relates to the identification of factors including adherence to one or more medical regimens to generate a health profile for a subject. In one embodiment, a medical regimen is a prophylactic regimen. Accordingly, the present invention features methods for identifying subjects who are at risk of developing the condition(s) for which one or more prophylactic medications are prescribed by detection of the factors and assessing the health profile disclosed herein. These factors or otherwise health profile are also useful for monitoring subjects undergoing treatments and therapies, and for selecting or modifying therapies and treatments to alternatives that would be efficacious in subjects having low rates of adherence when an acceptable alternative is available.

The risk of developing HIV can be assessed by measuring one or more of the factors described herein, and comparing the presence and values of the factors to reference or index values. Such a comparison can be undertaken with mathematical algorithms or formula in order to combine information from results of multiple individual factors and other parameters into a single measurement or index. Subjects identified as having an increased risk of HIV can optionally be selected to receive counseling, an increased frequency of monitoring, or treatment regimens, such as administration of therapeutic compounds. Subjects with HIV can optionally be selected to receive counseling or an increased frequency of monitoring relative to their individual health profile.

The factors of the present invention can thus be used to generate a health profile or signature of subjects: (i) who do not have and are not expected to develop HIV and/or (ii) who have or expected to develop HIV. The health profile of a subject can be compared to a predetermined or reference profile to diagnose or identify subjects at risk for developing HIV, to monitor the adherence to a prophylactic regimen, and to monitor the effectiveness of NRTI or other prophylactic pharmaceuticals. Data concerning the factors of the present invention can also be combined or correlated with other data or test results, such as, without limitation, measurements of clinical parameters or other algorithms for HIV.

Information obtained from the methods of the invention described herein can be used alone, or in combination with other information (e.g., age, race, sexual orientation, vital signs, blood chemistry, etc.) from the subject or from a biological sample obtained from the subject.

Various embodiments of the present invention describe mechanisms configured to monitor, track, and report levels of a prophylactic pharmaceutical in an individual at multiple time points. In one embodiment, the system allows for the collection of data for the presence of a metabolite associated with a prophylactic treatment regimen from multiple samples from an individual. The system can notify the user/evaluator about the likelihood of risk of developing the disorder or condition for which the prophylactic was prescribed when a change (i.e. increase or decrease) in the level of a metabolite associated with a prophylactic pharmaceutical is detected in subsequent samples from a single individual. For example, in some implementations, the system records the presence of a metabolite entered into the system by the user/evaluator or automatically recorded by the system on days 1, 2, 3 and 4 following taking a prophylactic pharmaceutical and applies algorithms to recognize patterns that predict the day at which the individual is at high risk of contracting a disorder in the absence of intervening administration of additional prophylactic. The algorithmic analysis, for example, may be conducted in a central (e.g., cloud-based) system. Data uploaded to the cloud can be archived and collected, such that learning algorithms refine analysis based upon the collective data set of all patients. In some implementations, the system combines quantified clinical features and physiology to aid in diagnosing risk objectively, early, and at least semi-automatically based upon collected data.

In some embodiments, the system is for personal use and tracking by the individual subject. In some embodiment, the data from the system is uploaded to a central system and a provider evaluates the data and makes a diagnosis or recommendation. Providers, in some implementations, may perform a live analysis through real-time data feed between a POCT system and a remote evaluator computing system.

The system has several advantages. The system can be in a form of a kit or an application in the context of an electronic device, such as an electronic hand held device or even a wearable data collection device for convenience. The system is beneficial to providers as well. The providers can evaluate adherence to a treatment regimen from home, during commute, or otherwise away from the office. Further, providers can approve of continued use of a prophylactic without an office visit provided the individual has been adhering to a prescribed regimen. Providers or the individuals themselves may also be altered by the system to transient lapses in a treatment adherence that would suggest an individual may be at increased risk.

In some implementations, the system is used to track an individual's ongoing progress. To enable such ongoing assessment, in some embodiments, applications for assessment may be made available for download to or streaming on a wearable data collection device via a network-accessible content store other content repositories, or other content collections. Content can range in nature from simple text, images, or video content or the like, to fully elaborated software applications (“apps”) or app suites. Content can be freely available or subscription based. Content can be stand-alone, can be playable on a wearable data-collection device based on its existing capabilities to play content (such as in-built ability to display text, images, videos, apps, etc., and to collect data), or can be played or deployed within a content-enabling framework or platform application that is designed to incorporate content from content providers. Content consumers, furthermore, can include individuals at risk of contracting HIV or their families as well as clinicians, physicians, and/or educators who wish to incorporate system modules into their professional practices.

In one embodiment, the system for assessing the risk of contracting HIV of the invention can be implemented on a cell phone, tablet computer, a desk top computer, and the likes. In some implementations, in addition to assessment, one or more modules of the system provide training mechanisms for supporting the individual's coping with HIV and its characteristics such as, in some examples, training mechanisms to assist in actions to take when receiving or providing First AID to an individual with HIV.

In one embodiment, the system of the invention can be in a medium that operates automatically behind the scenes in an electronic medical records database/software so that a notice automatically occurs if the data is designated to prompt an alert.

In another embodiment, the system of the invention can be in a format that encompasses “machine learning” so the process and comparator are update and improved as more information is entered and new analogs are developed.

### Administration

In one embodiment, the systems as described elsewhere herein can he administered to patients taking a prophylaxis. In one embodiment, the systems as described elsewhere herein can be administered to patients taking a pre-exposure prophylaxis. In one embodiment, the systems as described elsewhere herein can be administered to patients taking a NRTI such as TDF and/or FTC. In one embodiment, the systems as described elsewhere herein can be administered to patients taking a NRTI such as TAF and/or FTC. In one embodiment, the systems as described elsewhere herein can be administered to patients taking Truvada™.

In one embodiment, the systems of the invention are administered to a patient by a provider in a clinical setting during a visit. In another embodiment, the systems are used by the patient outside of a clinical setting. In one embodiment, a patient using the system outside of the clinical setting could inform a physician of the results. In one embodiment, a patient using the system outside of the clinical setting could do so independent of reporting the results to a physician.

### Biological Samples

Biological samples to be analyzed using the invention may be of any biological tissue or fluid containing the NRTI. Frequently the sample will be a “clinical sample” which is a sample derived from a patient. Typical samples for analysis include, but are not limited to, biological fluid samples such as sputum (a.k.a saliva), blood, plasma, milk, semen and urine.

Methods for collection of biological fluids from patients are well known in the art. In one embodiment, collection of a biological fluid for use in a lateral flow rapid visual NRTI test is with a sample cup or other receptacle. In one embodiment, a lateral flow device of the invention is inserted into a sample cup or other receptacle containing a biological fluid specimen. Receptacles appropriate for use in collecting biological fluid samples for use with the invention are not necessarily limited and are well known in the art. In one embodiment, a patient places an absorbent wick of a lateral flow device of the invention into their urine flow to collect the biological fluid for analysis. In one embodiment, a lateral flow device of the invention is inserted into an oral cavity and contacts the oral mucosa to collect the biological fluid for analysis.

In one embodiment, biological samples or aliquots of biological samples are shipped to a lab for analysis using a lab based test. In one embodiment, biological samples or aliquots of biological samples are frozen for shipment to a lab for analysis using a lab based test.

### Test Results

In one embodiment, a lateral flow device provides results within 1 to 5 minutes. In this embodiment, the results can be read by the patient or provider and interpreted. In one embodiment, the patient sample is analyzed using a lab based test and results are sent by confidential electronic record or by confidential fax back to the patient or provider. Other methods of providing results to providers and patients are well known.

In one embodiment, the results are used by a provider to monitor the adherence of a patient to a prescribed dosing schedule. In one embodiment, the test results are interpreted by a provider and used to inform a counseling strategy with the patient either in person or by phone, email, text message, or other communication medium. This includes but is not limited to a discussion with the patient, formulating a care plan, adjusting insurance coverage, addressing barriers to medication adherence, assigning an individual to check on compliance, using a digital solution such as text messaging to improve adherence, or a mechanical solution such as a pill dispenser that records and/or transmits data on pill consumption. Additionally, the provider can use this information to flag patients in which urine testing has shown that they are either not protected (e.g. urine TFV concentration <10 ng/mL, if using the LC-MS/MS based assay) or incompletely protected (e,g. urine TFV concentration between 10 and 1000 ng/mL, if using the LC-MS/MS based assay) from HIV acquisition based on their most recent urine TFV levels.

In one embodiment, the patient could use the system outside of a clinical setting. In one embodiment, the patient could use the system at the direction of a provider. In one embodiment, the patient could inform their provider of their results. This could include but is not limited to informing the provider after each individual test through a phone call, messaging, or digital app or performing multiple tests and providing the results to the provider at intermittent visits.

In an alternative embodiment, the patient could use the system independently of provider oversight. In this embodiment, the results could used by a patient to confirm the presence of a NRTI prior to an encounter wherein they are at risk of contracting HIV.

In one embodiment, testing can be performed daily. In one embodiment, testing can be performed before a high-risk encounter in which the patient is at risk of becoming HIV infected. In one embodiment, testing can be performed at a frequency determined by a provider or research director.

In one embodiment, a POCT of the invention can be used along with a handheld device. In one embodiment, a handheld device for use with a POCT of the invention analyzes the results of the POCT. In one embodiment, the analysis is performed using an electronic detection method incorporated into the handheld device. In one embodiment, the handheld device of the invention interfaces with a computer program. In one embodiment, a computer program is an application or web-based evaluation tool. In one embodiment, a user accesses a computer program to analyze, track, or visualize the test results. In one embodiment, a computer program for analyzing, tracking, or visualizing the test results from a POCT also serves to report test results to a physician or other party.

### Metabolites

In one embodiment, the system disclosed herein includes application of a biological fluid obtained from a test sample to a system for the detection of one or more metabolites that are associated with a pharmaceutical. In one embodiment, the pharmaceutical is used to treat a disease. In one embodiment, the pharmaceutical is used as a preventative measure. Such metabolites include, but are not limited to small molecules, metabolic products, degradation products, or related metabolites of one or more NRTIs.

In one embodiment, a pharmaceutical is comprised of one or more NRTIs. In one embodiment, the pharmaceutical is used to treat HIV infection. In one embodiment, the pharmaceutical is used to prevent HIV infection. Such metabolites include, but are not limited to small molecules, metabolic products, degradation products, or related metabolites of one or more NRTIs.

In one embodiment, the present disclosure relates to immunoassays for assessing (e.g., detecting or quantifying) at least one NRTI of interest in a test sample. In one embodiment, the invention relates to an immunoassay to detect TFV. In one embodiment, the invention relates to an immunoassay to detect FTC. In one embodiment, the invention relates to an immunoassay to detect both TFV and FTC.

Controls with respect to the presence or absence of the NRTI or concentration of the NRTI may be to metabolites abundant in the sample to be tested. In one embodiment, controls may be to markers abundant in at least one of urine, saliva, blood or plasma. As described elsewhere herein, comparison of the test patterns of the NRTI to be tested with those of the controls can be used to identify the presence of the NRTI. In this context, the control or control group is used for purposes of establishing proper use and function of the systems and assay of the invention. Therefore, mere detection of a NRTI of the invention without the requirement of comparison to a control group can be used to identify the presence of the NRTI. In this manner, the system according to the present invention may be used for qualitative (yes/no answer); semi-quantitative (−/+/++/+++/++++) or quantitative answer.

The concentration or level of a NRTI in urine is associated with plasma concentration levels of the NRTI. Thus, the concentration level of NRTIs in urine serves as a signpost for the increased or decreased risk of contracting HIV upon exposure that is afforded by the NRTI. For example using the LC-MS/MS based assay, a urine TFV concentration <10 ng/mL may indicate that a patient is at high risk of contracting HIV upon an exposure incident, whereas a urine TFV concentration between 10 and 1000 ng/mL may indicate that a patient is at some risk of contracting HIV upon an exposure incident and a urine TFV concentration >1000 ng/mL may indicate that a patient is at low risk of contracting HIV upon an exposure incident.

### Disease

In one embodiment, a person diagnosed with HIV may be prescribed a pharmaceutical comprising one or more NRTIs for treatment of HIV. In one embodiment, an individual at risk of contracting HIV may be prescribed a pharmaceutical comprising one or more NRTIs to be taken daily as a preventative measure to reduce the risk of contracting HIV from an exposure incident. Such an individual may be a relative of an individual diagnosed with HIV. Such an individual may be a long term care provider for an individual diagnosed with HIV. Such an individual may be a short term care provider for an individual diagnosed with HIV. Such an individual may be a residential or non-residential partner of an individual diagnosed with HIV. In certain cases, such an individual may participate in research involving HIV or pharmaceuticals for the treatment or prevention of HIV.

In one embodiment, the invention provides a system for quickly determining whether an individual has recently (e.g. within one week) taken a NRTI. In one embodiment, the test results can be used to determine whether an individual has taken a pharmaceutical comprising one or more NRTI as prescribed by a provider or research study manager. In one embodiment, the test results can be used to determine whether an individual is at high risk of contracting HIV upon an exposure incident.

In one aspect, the invention is useful because determination of an individual's level of compliance with a prescribed preventative or treatment plan can inform a physician as to future treatment plans for the individual. In one aspect, the invention is useful because determination of an individual's level of compliance with a research study can inform a researcher as to the validity of data gathered for the efficacy of a new NRTI pharmaceutical. For example, if an individual participating in a research study testing a new NRTI uses the invention and the test results indicate that the person has taken the NRTI as prescribed then confidence is provided for the research study results. Alternatively, if the test results indicate that the individual has not taken the NRTI as prescribed then the researcher may determine that the individual should be removed from the ongoing study.

In one embodiment, incentive methods may be provided to improve adherence to a prescription plan wherein an individual is incentivized in any manner to take a pharmaceutical comprising a NRTI and the invention is used to monitor adherence to the prescription plan. Incentive methods are well known in the art and include but are not limited to monetary compensation and gamification.

In one embodiment, the invention relates to urine assays for other medications, including other medications ultimately used as prophylactic or PrEP agents. In one embodiment, the invention relates to point of care assays for other medications, including other medications ultimately used as prophylactic or PrEP agents.

### Detecting an Analyte

The concentration of the analyte or metabolite in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a metabolite in a sample.

In one embodiment, the sample of the invention is a biological sample. The biological sample can originate from solid or fluid samples. Preferably the sample is a fluid sample. The sample of the invention may comprise urine, whole blood, blood serum, blood plasma, sweat, mucous, saliva, milk, semen and the like.

### Immunoassays

In one embodiment, the systems and methods of the invention can be performed in the form of various immunoassay formats, which are well known in the art. Immunoassays, in their most simple and direct sense, are binding assays involving binding between antibodies and antigen. Many types and formats of immunoassays are known and all are suitable for detecting the disclosed metabolites. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), fluorescence recovery/localization after photobleaching (FRAP/FLAP), a sandwich assay, a competitive assay, an immunoassay using a biosensor, an immunoprecipitation assay, an agglutination assay, a turbidity assay, a nephlelometric assay, etc.

In general, immunoassays involve contacting a sample suspected of containing a molecule of interest (such as the disclosed metabolites) with an antibody to the molecule of interest or contacting an antibody to a molecule of interest (such as antibodies to the disclosed metabolites) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed metabolites or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength.

There are two main types of immunoassays, homogeneous and heterogeneous. In homogeneous immunoassays, both the immunological reaction between an antigen and an antibody and the detection are carried out in a homogeneous reaction. Heterogeneous immunoassays include at least one separation step, which allows the differentiation of reaction products from unreacted reagents. A variety of immunoassays can be used to detect one or more of the proteins disclosed or incorporated by reference herein.

ELISA is a heterogeneous immunoassay, which can be used in the methods disclosed herein. The assay can be used to detect protein antigens in various formats. In the “sandwich” format the antigen being assayed is held between two different antibodies. In this method, a solid surface is first coated with a solid phase antibody. The test sample, containing the antigen e.g., a diagnostic protein), or a composition containing the antigen, such as a urine sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. A heterogeneous immunoassay, such as an ELISA, can be used to detect any of the proteins disclosed or incorporated by reference herein.

Homogeneous immunoassays include, for example, the Enzyme Multiplied Immunoassay Technique (EMIT), which typically includes a biological sample comprising the metabolites to be measured, enzyme-labeled molecules of the metabolites to be measured, specific antibody or antibodies binding the metabolites to be measured, and a specific enzyme chromogenic substrate. In a typical EMIT, excess of specific antibodies is added to a biological sample. If the biological sample contains the proteins to be detected, such proteins bind to the antibodies. A measured amount of the corresponding enzyme-labeled proteins is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the proteins disclosed or incorporated by reference herein.

In many immunoassays, as described elsewhere herein, detection of antigen is made with the use of antigens specific antibodies as detector molecules. However, immunoassays and the systems and methods of the present invention are not limited to the use of antibodies as detector molecules. Any substance that can bind or capture the antigen within a given sample may be used. Aside from antibodies, suitable substances that can also be used as detector molecules include but are not limited to enzymes, peptides, proteins, and nucleic acids. Further, there are many detection methods known in the art in which the captured antigen may be detected. In some assays, enzyme-linked antibodies produce a color change. In other assays, detection of the captured antigen is made through detecting fluorescent, luminescent, chemiluminescent, or radioactive signals. The system and methods of the current invention is not limited to the particular types of detectable signals produced in an immunoassay.

Immunoassay kits are also included in the invention. These kits include, in separate containers (a) monoclonal antibodies having binding specificity for the polypeptides used in the diagnosis of inflammation or the source of inflammation; and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may be a polyclonal immunoglobulin, protein A or protein G or functional fragments thereof, which may be labeled prior to use by methods known in the art. In several embodiments, the immunoassay kit includes two, three or four of: antibodies that specifically bind a protein disclosed or incorporated herein.

In one embodiment, the immunoassay kit of the invention can comprise (a) a sample pad, (b) a conjugated label pad, the conjugated label pad having a detectable label, a portion of the conjugated label pad and a portion of the sample pad forming a first interface, (c) a lateral-flow assay comprising a membrane, a portion of the membrane and a portion of the conjugated label pad forming a second interface, and (d) at least one antibody bound to the membrane, the first interface allowing fluid to flow from the sample pad to the conjugated label pad and contact the detectable label wherein the metabolite present in the sample forms an metabolite-conjugated label complex, the second interface allowing fluid to flow from the conjugated label pad to the membrane and to contact the at least one membrane-bound antibody to form to an metabolite-antibody complex and cause the detectable label to form a detectable signal.

In one embodiment, the immunoassay kit of the invention includes an additional component including but not limited to one or more of instructional material and sample collection receptacles. In one embodiment, the kit of the invention includes a single immunoassay system. In one embodiment, the kit of the invention includes more than one immunoassay system.

In one embodiment, the kit of the invention includes a handheld device. In one embodiment, the kit includes a system for or access to a computer software for analyzing, recording, monitoring, tracking an reporting the results of the POCT of the invention.

### Mass Spectrometry and Chromatography

In one embodiment, the method of detection is a lab based test. In one embodiment, the lab based test is a semi-quantitative liquid chromatography-tandem mass spectrometry (LC-MS/MS) urine assay.

In one embodiment, the systems and methods of the invention can be performed in the form of various mass spectrometry (MS) or chromatography formats, which are well known in the art. As such, the levels of metabolites present in a sample can be determined by mass spectrometry. Generally, any mass spectrometric techniques that can obtain precise information on the mass of peptides, and preferably also on fragmentation and/or (partial) amino acid sequence of selected peptides, are useful herein. Suitable peptide MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein.

The terms “mass spectrometry” or “MS” as used herein refer to methods of filtering, detecting, and measuring ions based on their mass-to-charge ratio. or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). For examples see U.S. Pat. Nos. 6,204,500, 6,107,623, 6,268,144, 6,124,137; Wright et al.. 1999, Prostate Cancer and Prostatic Diseases 2: 264-76; Merchant et al., 2000, Electrophoresis 21: 1164-67, each of which is hereby incorporated by reference in its entirety, including all tables, figures, and claims. Mass spectrometry methods are well known in the art and have been used to quantify and/or identify biomolecules, such as proteins and hormones (Li et al., 2000, Tibtech. 18:151-160; Starcevic et. al., 2003, J. Chromatography B, 792: 197-204; Kushnir et. al., 2006, Clin. Chem. 52:120-128; Rowley et al., 2000, Methods 20: 383-397; Kuster et al., 1998. Curr. Opin. Structural Biol. 8: 393-400). Further, mass spectrometric techniques have been developed that permit at least partial de novo sequencing of isolated proteins (Chait et al., 1993, Science, 262:89-92; Keough et al., 1999, Proc. Natl. Acad. Sci. USA. 96:7131-6; Bergman, 2000, EXS 88:133-44). Various methods of ionization are known in the art. For examples, Atmospheric Pressure Chemical Ionization (APCI) Chemical Ionization (CI) Electron Impact (EI) Electrospray Ionization (ESI) Fast Atom Bombardment (FAB) Field Desorption/Field Ionization (FD/FI) Matrix Assisted Laser Desorption Ionization (MALDI) and Thermospray Ionization (TSP).

The levels of metabolites present in a sample can be determined by MS such as matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; tandem mass spectrometry (e.g., MS/MS, MS/MS/MS etc.); electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)¹; atmospheric pressure photoionization mass spectrometry (APPI-MS); APPI-MS/MS; APPI-(MS)ⁿ; liquid chromatography-mass spectrometry (LC-MS), gas chromatography-mass spectrometry (GC-MS); high performance liquid chromatography-mass spectrometry (HPLC-MS); capillary electrophoresis-mass spectrometry; and nuclear magnetic resonance spectrometry. Peptide ion fragmentation in tandem MS (MS/MS) arrangements may be achieved using manners established in the art, such as, e.g., collision induced dissociation (CID). See for example, U.S. Patent Application Nos: 20030199001, 20030134304, 20030077616, which are herein incorporated by reference in their entirety. Such techniques may be used for relative and absolute quantification and also to assess the ratio of the metabolite according to the invention with other metabolites that may be present. These methods are also suitable for clinical screening, prognosis, monitoring the results of therapy_{;} identifying patients most likely to respond to a particular therapeutic treatment, for drug screening and development, and identification of new targets for drug treatment.

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937, and U.S. Pat. No. 5,045,694. In SELDI, the substrate surface is modified so that it is an active participant in the desorption process. In one variant, the surface is derivatized with adsorbent and/or capture reagents that selectively bind the metabolite of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. SELDI is a powerful tool for identifying a characteristic “fingerprint” of proteins and peptides in body fluids and tissues for a given condition, e.g. drug treatments and diseases. This technology utilizes protein chips to capture proteins/peptides and a time-of-flight mass spectrometer (tof-MS) to quantitate and calculate the mass of compounds ranging from small molecules and peptides of less than 1,000 Da up to proteins of 500 kDa. Quantifiable differences in protein/peptide patterns can be statistically evaluated using automated computer programs which represent each protein/peptide measured in the biofluid spectrum as a coordinate in multi-dimensional space. The SELDI system also has a capability of running hundreds of samples in a single experiment. In addition, all the signals from SELDI mass spectrometry are derived from native proteins/peptides (unlike some other proteomics technologies which require protease digestion), thus directly reflecting the underlying physiology of a given condition.

In MALDI and SELDI, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material. For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4th ed. Vol. 15 (John Wiley Sons, New York 1995), pp. 1071-1094. Detection and quantification of the metabolite will typically depend on the detection of signal intensity. For example, in certain embodiments, the signal strength of peak values from spectra of a first sample and a second sample can be compared (e.g., visually, by computer analysis etc.), to determine the relative amounts of particular metabolite. Software programs such as the Biomarker Wizard program (Ciphergen Biosystems, Inc., Fremont, Calif.) can be used to aid in analyzing mass spectra. The mass spectrometers and their techniques are well known to those of skill in the art.

In an embodiment, detection and quantification of metabolites by mass spectrometry may involve multiple reaction monitoring (MRM), such as described among others by Kuhn et al. 2004 (Proteomics 4: 1175-86).

In an embodiment, MS peptide analysis methods may be advantageously combined with upstream peptide or protein separation or fractionation methods, such as for example with the chromatographic and other methods described herein below.

Chromatography can also be used for measuring metabolites. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably high-performance liquid chromatograph7 (HPLC). While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993.

Exemplary types of chromatography include, without limitation, HPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like.

In an embodiment, chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring metabolites in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FEE), etc.

### Point-of-Use Devices

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin. In some point-of use devices, assays are performed with test strips in which a specific binding pair member is attached to a mobilizable material (such as a metal sol or beads made of latex or glass) or an immobile substrate (such as glass fibers, cellulose strips or nitrocellulose membranes). Other point-of use devices may comprise optical biosensors, photometric biosensors, electrochemical biosensor, or other types of biosensors. Suitable biosensors in point-of-use devices for performing methods of the invention include “cards” or “chips” with optical or acoustic readers. Biosensors can be configured to allow the data collected to be electronically transmitted to the physician for interpretation and thus can form the basis for e-medicine, where diagnosis and monitoring can be done without the need for the patient to be in proximity to a physician or a clinic.

Detection of a metabolite in a sample can be carried out using a sample capture device, such as a lateral flow device (for example a lateral flow test strip) that allows detection of one or more metabolites, such as those described herein.

The test strips of the present invention include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. Nos. 5,229,073, 5,591,645; 4,168,146; 4,366,241; 4,855,240; 4,861,711; 5,120,643 (each of which are herein incorporated by reference). Multiple zone lateral flow test strips are disclosed in U.S. Pat. Nos. 5,451,504, 5,451,507, and U.S. Pat. No. 5,798,273 (incorporated by reference herein). U.S. Pat. No. 6,656,744 (incorporated by reference) discloses a lateral flow test strip in which a label binds to an antibody through a streptavidin-biotin interaction.

Flow-through type assay devices were designed, in part, to obviate the need for incubation and washing steps associated with dipstick assays. Flow-through immunoassay devices involve a capture reagent (such as one or more antibodies) bound to a porous membrane or filter to which a liquid sample is added. As the liquid flows through the membrane, target analyte (such as protein) binds to the capture reagent. The addition of sample is followed by (or made concurrent with) addition of detector reagent, such as labeled antibody (e.g., gold-conjugated or colored latex particle-conjugated protein). Alternatively, the detector reagent may be placed on the membrane in a manner that permits the detector to mix with the sample and thereby label the analyte. The visual detection of detector reagent provides an indication of the presence of target analyte in the sample. Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030,546. Migration assay devices usually incorporate within them reagents that have been attached to colored labels, thereby permitting visible detection of the assay results without addition of further substances. See, for example, U.S. Pat. No. 4,770,853; PCT Publication No. WO 88/08534.

There are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte. Additional examples of lateral flow tests for large analytes are disclosed in the following patent documents: U.S. Pat. Nos. 4,168,146; 4,366,241; 4,855,240; 4,861,711; and 5,120,643; WO 97/06439; WO 98/36278; and WO 08/030,546.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more proteins disclosed herein. Thus these zones can be viewed as functional sectors or functional regions within the test device.

In general, a fluid sample is introduced to the strip at the proximal end of the strip, for instance by clipping or spotting. A sample is collected or obtained using methods well known to those skilled in the art. The sample containing the particular proteins to be detected may be obtained from any biological source. In a particular example, the biological source is urine. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

In some embodiments, porous solid supports, such as nitrocellulose, described elsewhere herein are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm. (i.e., 100 to 250 sec./4 cm), about 37.5 to 62.5 sec/cm (i.e., 150 to 250 sec/4 cm), or about 50 to 62.5 sec/cm (i.e., 200 to 250 sec/4 cm).

Another common feature to be considered in the use of assay devices is a means to detect the formation of a complex between an analyte (such as one or more proteins described herein) and a capture reagent (such as one or more antibodies). A detector (also referred to as detector reagent) serves this purpose. A detector may be integrated into an assay device (for example includes in a conjugate pad), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area.

### Flow-Through Device Construction and Design

A flow-through device involves a capture reagent (such as one or more antibodies) immobilized on a solid support, typically, microtiter plate or a membrane (such as, nitrocellulose, nylon, or PVDF). In a simple representative format, the membrane of a flow-through device is placed in functional or physical contact with an absorbent layer, which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more proteins described herein) can specifically bind to the immobilized capture reagent (such as one or more antibodies). Where detection of an analyte-capture reagent complex is desired, a detector reagent (such as labeled antibodies that specifically bind one or more proteins) can be added with the sample or a solution containing a detector reagent can be added subsequent to application of the sample. If an analyte is specifically bound by capture reagent, a characteristic attributable to the particular detector reagent can be observed on the surface of the membrane. Optional wash steps can be added at any time in the process, for instance, following application of the sample, and/or following application of a detector reagent.

### Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,258,548; 6,699,722; 6,368,876 and 7,517,699, each of which is incorporated by reference.

Many lateral flow devices are one-step lateral flow assays in which a biological fluid is placed in a sample area on a bibulous strip (though non-bibulous materials can be used, and rendered bibulous, e.g., by applying a surfactant to tile material), and allowed to migrate along the strip until the liquid comes into contact with a specific binding partner (such as an antibody) that interacts with an analyte (such as one or more proteins) in the liquid. Once the analyte interacts with tile binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more proteins) in tile liquid. The test strips can also incorporate control indicators, which provide a signal that the test has adequately been performed, even if a positive signal indicating the presence (or absence) of an analyte is not seen on the strip.

Lateral flow devices have a wide variety of physical formats that are equally well known in the art. Any physical format that supports and/or houses the basic components of a lateral flow device in the proper function relationship is contemplated by this disclosure.

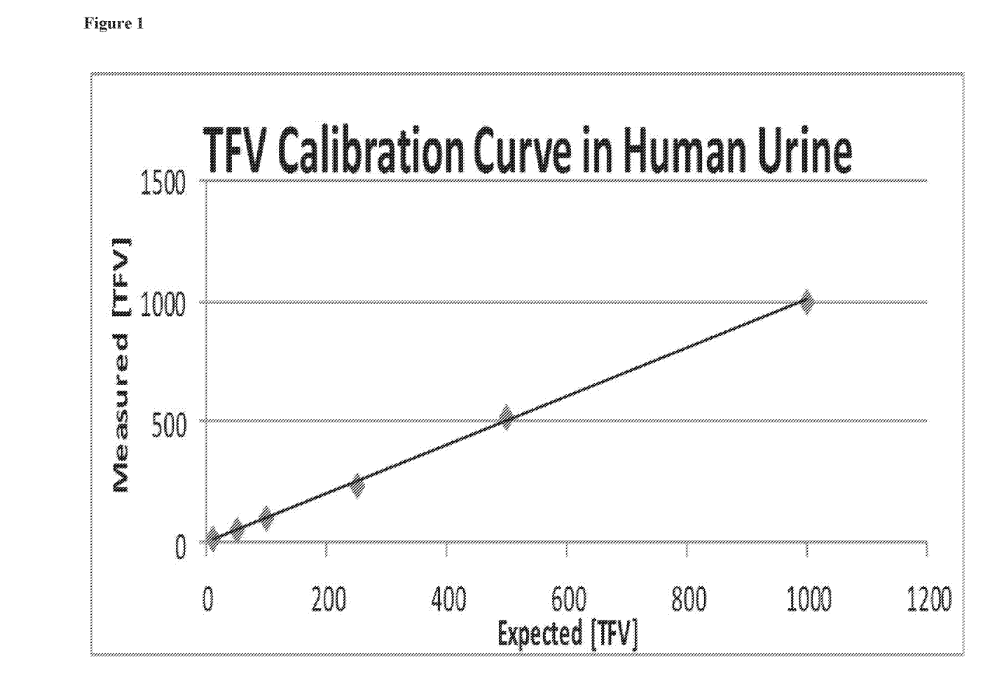

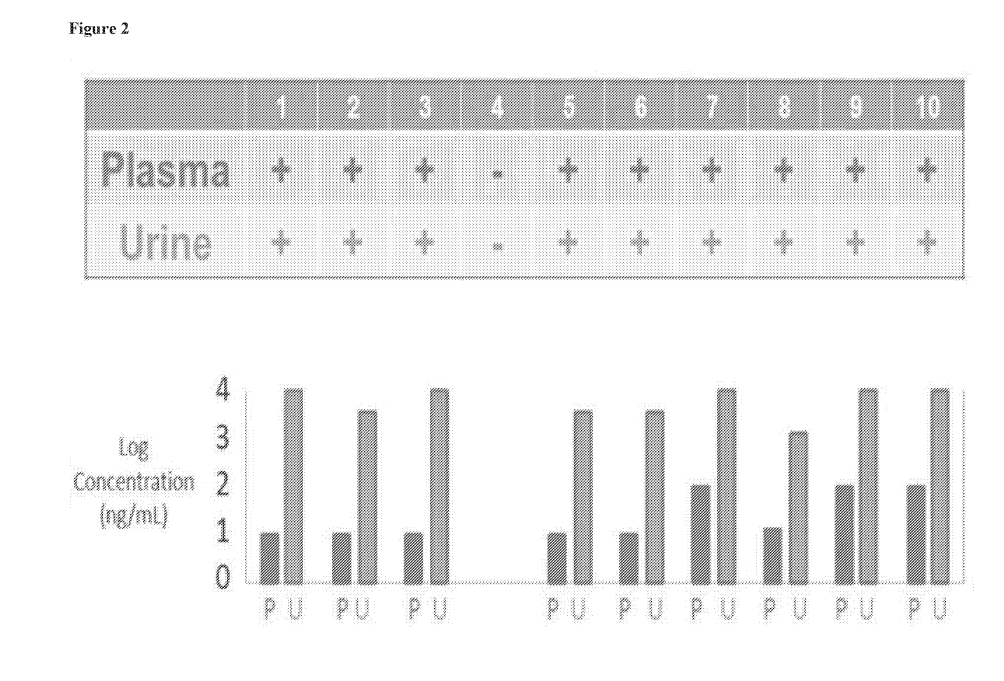

The basic components of a particular embodiment of a lateral flow device are illustrated in FIGS. 1 and 2 which comprise a sample pad, a conjugate pad, a migration membrane, and an absorbent pad.

The sample pad (such as the sample pad in FIGS. 1 and 2) is a component of a lateral flow device that initially receives the sample, and may serve to remove particulates from the sample. Among the various materials that may be used to construct a sample pad (such as glass fiber, woven fibers, screen, non-woven fibers, cellosic fibers or paper) or a cellulose sample pad may be beneficial if a large bed volume is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

With respect to the migration membrane, the types of membranes useful in a lateral flow device include but are not limited to nitrocellulose (including pure nitrocellulose and modified nitrocellulose) and nitrocellulose direct cast on polyester support, polyvinylidene fluoride, or nylon).

The conjugate pad (such as conjugate pad in FIGS. 1 and 2) serves to, among other things, hold a detector reagent. Suitable materials for the conjugate pad include glass fiber, polyester, paper, or surface modified polypropylene.

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypmpylmethyl cellulose, SDS, Brij and β-lactose. A mixture of two or more release agents may be used in any given application.

With respect to the absorbent pad, the pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers), One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

In operation of the particular embodiment of a lateral flow device, a fluid sample containing an analyte of interest, such as one or more proteins described herein, is applied to the sample pad. In some examples, the sample may be applied to the sample pad by dipping the end of the device containing the sample pad into the sample (such as urine) or by applying the sample directly onto the sample pad.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the proteins described herein). For example, a protein analyte may bind to a labeled (e.g., gold-conjugated or colored latex particle-conjugated) antibody contained in the conjugate pad. The analyte complexed with the detector reagent may subsequently flow to the test line where the complex may further interact with an analyte-specific binding partner (such as an antibody that binds a particular protein, an anti-hapten antibody, or streptavidin), which is immobilized at the proximal test line. In some examples, a protein complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line, is detected. The control line may contain an immobilized, detector-reagent-specific binding partner, which can bind the detector reagent in the presence or absence of the analyte. Such binding at the control line indicates proper performance of the test, even in the absence of the analyte of interest.

In one embodiment, the control line detects the presence of one of IgG, IgD IgA or another constituent of urine. In one embodiment, the control line detects the presence of one of glycoproteins, secretory IgA, lactoferrin, lysozyme and peroxidase, or another constituent of saliva.

The test results may be visualized directly, or may be measured using a reader (such as a scanner). The reader device may detect color, fluorescence, luminescence, radioactivity, or any other detectable marker derived from the labeled reagent from the readout area (for example, the test line and/or control line).

In another embodiment of a lateral flow device, there may be a second (or third, fourth, or more) test line located parallel or perpendicular (or in any other spatial relationship) to the test line in the test result. The operation of this particular embodiment is similar to that described elsewhere herein with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

In one embodiment, a comparison of the control line to the test line yields the test result from the diagnostic system of the invention. In some instances, a valid result occurs when the control line is detected at a higher intensity level than the test line. For example, a valid result occurs when the control line is at least 5% or more, for example, 10%, 20%, 30%, 40%, 50%, 60%, 70%, 80%, 90%, 100% or more darker than the test line. In some instances, a valid result occurs when the control line is at least 0.5 fold or more, for example, 1 fold, 2 fold, 3 fold, 4 fold, 5 fold, 6 fold, 7 fold, 8 fold, 9 fold, 10 fold or more darker than the test line.

### Point of Care Diagnostic and Risk Assessment Systems

The system of the invention can be applied to a point-of-care scenario. U.S. Pat. Nos. 6,267,722, 6,394,952 and 6,867,051 disclose and describe systems for diagnosing and assessing certain medical risks, the contents of which are incorporated herein. The systems are designed for use on site at the point of care, where patients are examined and tested, as well as for operation remote from the site. The systems are designed to accept input in the form of patient data, including, but not limited to biochemical test data, physical test data, historical data and other such data, and to process and output information, such as data relating to a medical diagnosis or a disease risk indicator. The patient data may be contained within the system, such as medical records or history, or may be input as a signal or image from a medical test or procedure, for example, immunoassay test data, blood pressure reading, ultrasound, X-ray or MRI, or introduced in any other form. Specific test data can be digitized, processed and input into the medical diagnosis expert system, where it may be integrated with other patient information. The output from the system is a disease risk index or medical diagnosis.

Point of care testing refers to real time diagnostic testing that can be done in a rapid time frame so that the resulting test is performed faster than comparable tests that do not employ this system. For example, the exemplified immunoassay disclosed and described herein can be performed in significantly less time than the corresponding ELISA assay, e.g., in less than half an hour. In addition, point of care testing refers to testing that can be performed rapidly and on site, such as in a doctor's office, at a bedside, in a stat laboratory, emergency room or other such locales, particularly where rapid and accurate results are required.

In an exemplary embodiment, a point of care diagnostic and risk assessment system includes a reader for reading patient data, a test device designed to be read in the reader, and software for analysis of the data. A test strip device in a plastic housing is designed for use with the reader, optionally including a symbology, such as an alphanumeric character bar code or other machine-readable code, and software designed for analysis of the data generated from the test strip are also provided.

In one embodiment, a reader refers to an instrument for detecting and/or quantitating data, such as on test strips. The data may be visible to the naked eye, but does not need to be visible. Such readers are disclosed and described in the above-incorporated U.S. Pat. Nos. 6,267,722, 6,394,952 and 6,867,051. A reflectance reader refers to an instrument adapted to read a test strip using reflected light, including fluorescence, or electromagnetic radiation of any wavelength. Reflectance can be detected using a photodetector or other detector, such as charge coupled diodes (CCD). An exemplary reflectance reader includes a cassette slot adapted to receive a test-strip, light-emitting diodes, optical fibers, a sensing head, including means for positioning the sensing head along the test strip, a control circuit to read the photodetector output and control the on and off operation of the light-emitting diodes, a memory circuit for storing raw and/or processed data, and a photodetector, such as a silicon photodiode detector. It will be appreciated that a color change refers to a change in intensity or hue of color or may be the appearance of color where no color existed or the disappearance of color.

In one embodiment, a sample is applied to a diagnostic immunoassay test strip, and colored or dark bands are produced. The intensity of the color reflected by the colored label in the test region (or detection zone) of the test strip is, for concentration ranges of interest, directly proportional or otherwise correlated with an amount of analyte present in the sample being tested. The color intensity produced is read, in accordance with the present embodiment, using a reader device, for example, a reflectance reader, adapted to read the test strip. The intensity of the color reflected by the colored label in the test region (or detection zone) of the test strip is directly proportional to the amount of analyte present in the sample being tested. In other words, a darker colored line in the test region indicates a greater amount of analyte, whereas a lighter colored line in the test region indicates a smaller amount of analyte. The color intensity produced, i.e., the darkness or lightness of the colored line, is read using a reader device, for example, a reflectance reader, adapted to read the test strip.

A reflectance measurement obtained by the reader device is correlated to the presence and/or quantity of analyte present in the sample. The reader takes a plurality of readings along the strip, and obtains data that are used to generate results that are an indication of the presence and/or quantity of analyte present in the sample. The system may correlate such data with the presence of a disorder, condition or risk thereof.

As mentioned elsewhere herein, in addition to reading the test strip, the reader may (optionally) be adapted to read a symbology, such as a bar code, which is present on the test strip or housing and encodes information relating to the test strip device and/or test result and/or patient, and/or reagent or other desired information. Typically the associated information is stored in a remote computer database, but can be manually stored. Furthermore, the symbology can be imprinted when the device is used and the information encoded therein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### System for Detecting NRTI in a Urine Sample

TDF/FTC (Truvada™) is approved for pre-exposure prophylaxis (PrEP) for HIV infection. Adherence is critical for the success of PrEP, but current adherence measurements (self-report) and plasma tenofovir (TFV) levels are inadequate tools for real time adherence monitoring. Our goal was to develop and validate a urine assay for the measurement of TDF levels to objectively monitor adherence to PrEP.

### The Methods are Now Described

3 cohort studies were conducted to assess a system for detection of the active metabolite tenofovir (TFV) of the prodrug nucleotide reverse transcriptase inhibitor (NRTI) tenofovir disoproxil fumarate (TDF). Cohort 1: a cross sectional study of 10 HIV positive subjects with undetectable HIV viral loads on a TDF-based regimen; Cohort 2: a single dose study of Truvada in 10 healthy subjects to evaluate TFV clearance in plasma and urine over 7 days; Cohort 3: a 16 week study of 10 HIV negative subjects receiving daily PrEP to evaluate concordance between plasma and urine over time.

### The Experimental Results are Now Described

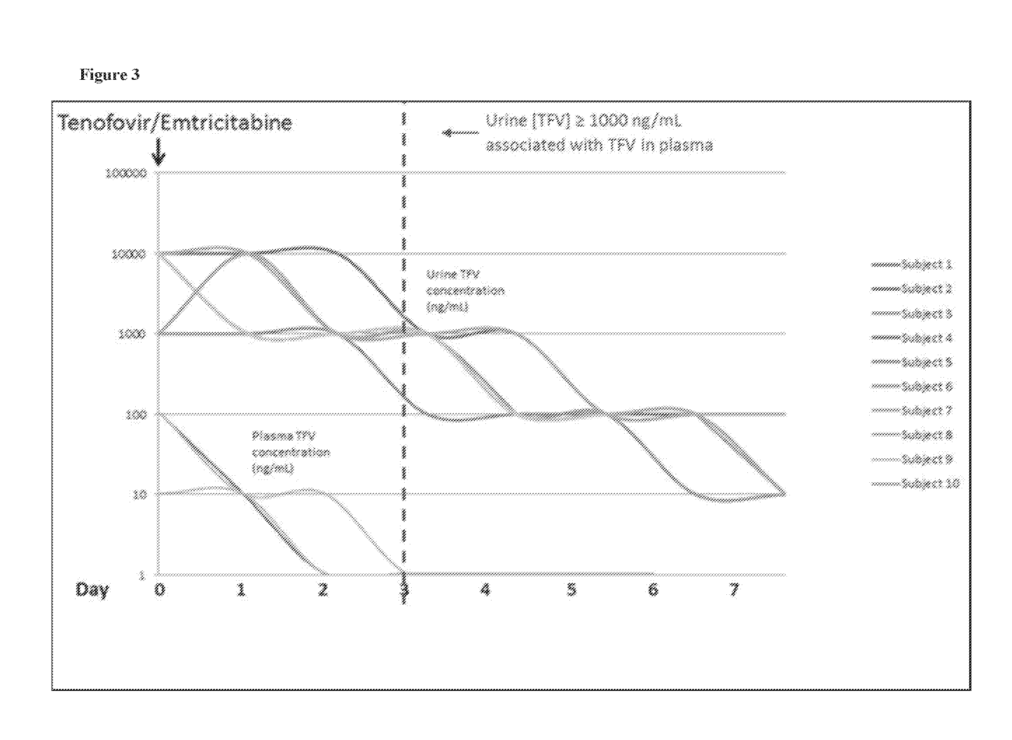

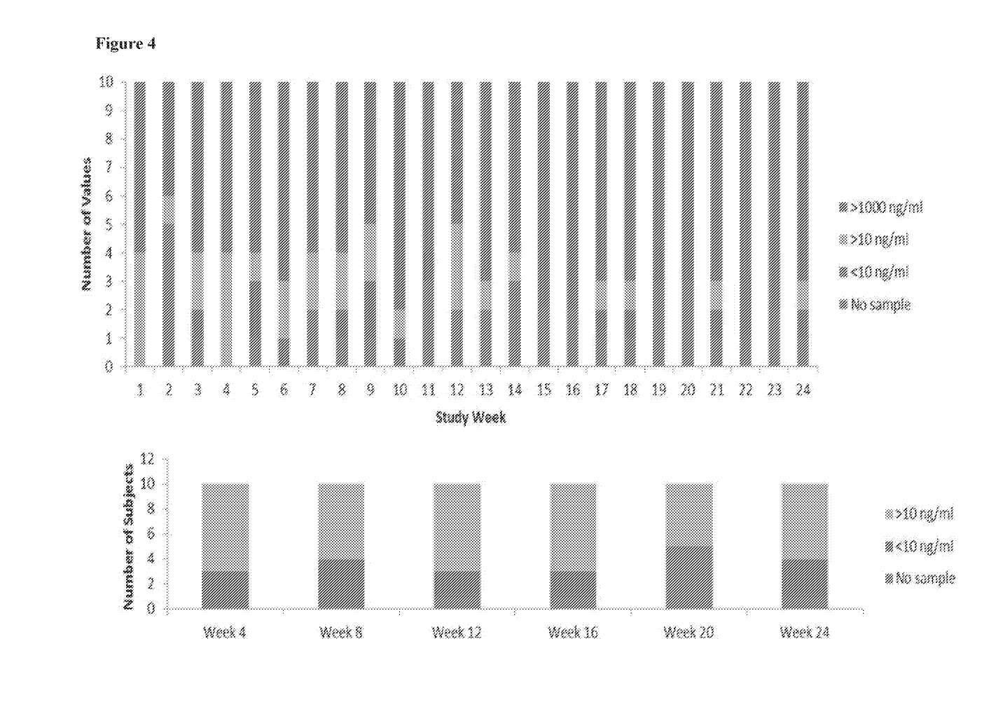

A semi-quantitative liquid chromatography-tandem mass spectrometry (LC-MS/MS) urine assay was developed for detection of TFV with high sensitivity/specificity. FIG. 1 demonstrates the ability of this assay to determine TFV concentrations in log categories between <10 ng/ml to >10,000 ng/ml. To evaluate the qualitative relationship of urine TFV levels to plasma levels, the levels of TFV in the urine and plasma from Cohort 1 was evaluated. FIG. 2 shows that there is 100% concordance between presence of TFV in plasma and urine (PPV 100%, 95% CI, 0.63-1.0; NPV 100%, 95% CI, 0.05-1.0). In addition, the TFV concentration was 3-4 logs higher in urine than plasma (FIG. 2). In cohort 2, TFV was detected for >7 days in urine and 2-4 days in plasma after a single dose of Truvada™ (FIG. 3). Urine TFV was cleared in a log-linear fashion, with a direct correlation of urine levels to time since last dose. The urine assay was 2 logs more sensitive than serum over 7 days (FIG. 3). In cohort 3, TFV was detected in 93% of urine samples (concentration range: >10 to >10,000 ng/ml) and 74% of plasma samples (concentration range: >10 ng/ml to >100 ng/ml) (FIG. 4). Urine TFV concentration >1000 ng/ml was highly predictive of presence of TFV in plasma (>10 ng/ml) (PPV 0.88, 95% CI, 0.69-0.97; NPV 0.88, 95% CI, 0.47-0.99), suggesting that the urine assay could be used to distinguish between recent adherence as defined by a dose of TFV within 48 hours (>1000 ng/ml), low adherence (>10 to >100 ng/ml), and non-adherence as defined by last dose more than one week prior (<10 ng/ml).

### Example 2

### Urine Assay Development

Antiretroviral concentrations in urine are potentially useful in monitoring adherence to PrEP. Although clinical data is limited, lamivudine levels in urine have been used as a means of monitoring antiretroviral adherence. Due to its short half-life of 5 to 7 hours, lamivudine was largely absent from the urine 24 hours after a single dose. The authors determined that a lamivudine concentration of 0.035 mg/mg creatinine or less at 48 hours was suggestive of a missed dose the previous day (Kumar 2006). Tenofovir is a more attractive drug to be used for monitoring adherence as it has a plasma half-life of 17 hours and intracellular half-life of 150 hours (Hawkins 2005), which allows the detection in the urine for several days. Our preliminary data demonstrate that TFV levels can be reliably measured in urine, that urine TFV correlates well with plasma concentrations, and that TFV detection in urine reflects medication usage over a window of one to at least seven days after oral TDF/FTC ingestion.

There is no standard adherence measurement that provides real time evaluation of adherence in patients receiving TDF/FTC to prevent HIV infection. Urine assays used for TDM have been shown to have clear benefit in improving adherence in several different fields, as described above, with very little downside when used as an adjunct to standard clinical assessment. In fact, in patients with refractory hypertension, a large study found that when patients were informed of their undetectable drug levels and provided additional counseling, blood pressure control was markedly improved without increasing treatment intensity (Brinker 2014). Urine TFV assessment can quickly provide information about whether someone is taking PrEP at all, and whether someone is taking PrEP well enough to protect them from HIV infection at the time of testing. Testing has shown that patients are either not protected (urine TFV concentration <10 ng/mL) or incompletely protected (urine TFV concentration between 10 and 1000 ng/mL) from HIV acquisition based on their most recent urine TFV levels.

Other PK-based measures of adherence are being studied in ongoing trials, the ultimate effectiveness of a urine-based screen for adherence and its usefulness in the clinical setting derive from three innovative aspects of this assay and study design.

1. Specific window period of the urine TFV assay. Urine TFV assessment fills a gap left by plasma/intracellular and hair assessments by providing information about medication adherence over at least a one-week period: single plasma concentrations only reflect a small window of exposure (approximately 2-3 days) (Nettles 2006; Clevenber 2002; Wertheimer 2006), whereas hair analysis and intracellular concentrations reflect average drug exposures over weeks to months (Liu 2014; Hawkins 2005).

2. Noninvasive nature of the urine assay. The urine TFV concentration may be an ideal adherence marker as it is preliminarily highly acceptable to individuals at risk of contracting HIV (Abstr. 975. CROI 2015). Especially among young men who have sex with men, the most at-risk group with respect to risk of HIV acquisition, urine collection provides advantages over blood draw for plasma or intracellular concentration, finger stick for DBS assessment, and pubic/scalp hair sampling for hair analysis. Adolescents demonstrate a significant preference for non-blood draw or needle-requiring assays in HIV testing (50.5% chose rapid oral swab vs. 30.3% chose traditional venipuncture vs. 19.2% chose rapid fingerstick blood test) and are likely to prefer urine collection to methods that require needles especially in the setting of weekly or biweekly monitoring for adherence.

3. A urine assay lends itself well to the development of a point-of-care assay, and we have applied for additional funding to that end. This study represents the ability to obtain proof-of-concept of the use of urine testing to improve adherence in order to inform ongoing efforts to turn this into a point-of-care test. This assay is sensitive and specific for TFV, does not require specific skills, is available at low-cost, and simple to collect and process. If this assay is acceptable to this population, it can be used in groups at risk of HIV infection (heterosexual women and men, intravenous drug users, serodiscordant couples, etc.) to enhance adherence and further improve HIV prevention efforts. Results of urine monitoring could potentially be used, much like viral load testing in HIV-positive patients, to engage patients in larger questions of risk awareness and stigma around use of PrEP.

A semi-quantitative liquid chromatography-tandem mass spectrometry (LC-MS/MS) urine assay with high sensitivity and specificity for TFV allowed us to determine TFV concentrations in log categories between <10 ng/L to >10,000 ng/mL. Following protein precipitation (0.1 mL human plasma or urine or diluted urine sample) using 100% acetonitrile with 0.1% formic acid, containing deuterated internal standard (2H6-tenofovir, 50 ng/mL), the analytes were separated using gradient mobile phase on a reverse phase C18 column and analyzed by MS/MS (AB Sciex API4000). The multiple reaction monitoring of m/z 288.3 to 176.3 for tenofovir and m/z 294.3 to 182.2 for 2H6-tenofovir was used for analysis. LC-MS/MS parameters for tenofovir and internal standard (2H6-tenofovir, 50 ng/mL in 0.1% formic acid in Acetonitrile) were optimized in positive electro-spray ionization mode and the separation was achieved with a Phenomenex Kinetex F5 column (2.6 μ, 100 A□, 4.6×50 mm). The method was found to be linear over the range of 10-1000 ng/mL with the limit of detection of 5 ng/mL. Flow rate was 0.7 ml/min and total chromatographic run-time was 3.0 minutes/sample.

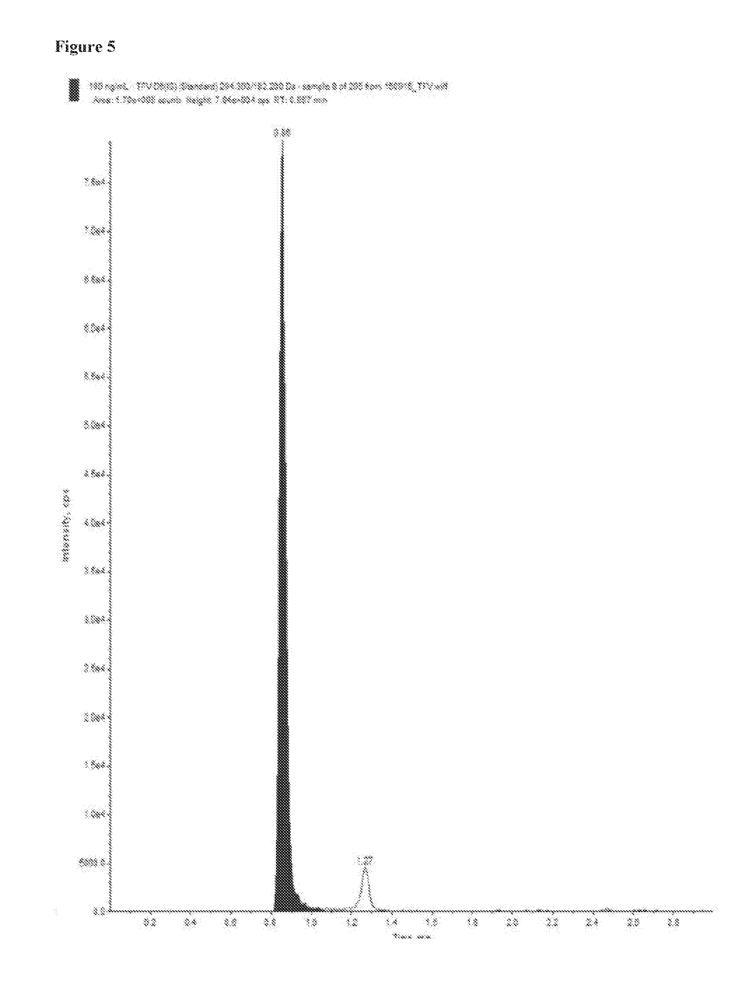

Standards (10, 100, 1000 and 10,000 ng/mL in human plasma), blanks (double blank and internal standard blank) and unknown plasma and urine samples were thawed to room temperature. One hundred μL of all standards and blanks were put into appropriate wells on a 96 well plate. Diluted unknown urine samples (95 μL blank plasma +5 μL of unknown urine samples, 20-fold dilution) or undiluted plasma samples were mixed with 400 μL of IS solution in a 96 well plate. The plate was vortexed for 30 seconds at high speed and centrifuged for 30 minutes at 4000 rpm. Supernatant was transferred to a fresh 96 well plate and a 10 μL aliquot was injected for LC-MS/MS analysis from autosampler. When the diluted sample had a concentration below 10 ng/mL, the samples were analyzed undiluted to measure the final concentration. When the concentration of TFV in urine samples exceeded 1,000 ng/mL, samples were diluted 50-fold with blank urine and reanalyzed. A representative chromatogram of tenofovir (100 ng/mL in human plasma) and internal standard is shown in FIG. 5.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for detecting a metabolite in a patient, the method comprising:
a) administering an effective amount of a Nucleoside Reverse Transcriptase Inhibitor (NRTI) to the patient;
b) obtaining a biological sample from the patient;
c) detecting whether the metabolite is present in the sample; and
d) determining adherence to a treatment or prophylactic regimen in the patient.

2. The method of claim 1, wherein the metabolite is tenofovir (TFV).

3. The method of claim 1, wherein the NRTI is selected from the group consisting of Tenofovir Disoproxil Fumarate (TDF), Emtricitabine (FTC), and Tenofovir Alafenamide (TAF), or derivatives thereof or combinations thereof.

4. The method of claim 3, wherein the NRTI is TAF.

5. The method of claim 3, wherein the NRTI is TDF.

6. The method of claim 3, wherein the NRTI is FTC.

7. The method of claim 3, wherein the NRTI is a combination of TDF/FTC.

8. The method of claim 3, wherein the NRTI is a combination of TAF/FTC.

9. The method of claim 1, wherein in step c), the metabolite is detected by immunoassay or spectrometry.

10. The method of claim 9, wherein the spectrometry is selected from the group consisting of matrix-assisted laser desorption/ionization time-of-flight (MALDI-TOF) mass spectroscopy (MS), MALDI-TOF post-source-decay (PSD), MALDI-TOF/TOF, surface-enhanced laser desorption/ionization time-of-flight (SELDI-TOF) MS, tandem MS, electrospray ionization MS (ESI-MS), ESI-MS/MS, ESI-MS/(MS)n, ESI 3D ion trap MS, ESI linear (2D) MS, ESI triple quadrupole MS, ESI quadrupole orthogonal TOF (Q-TOF), ESI Fourier transform MS, desorption/ionization on silicon (DIOS), secondary ion MS (SIMS), atmospheric pressure chemical ionization MS (APCI-MS), APCI-MS/MS, atmospheric pressure photoionization MS (APPI-MS); APPI-MS/MS, APCI-(MS)¹, liquid chromatography MS(LC-MS), semi-quantitative liquid chromatography-tandem (LC-MS/MS), gas chromatography-MS (GC-MS), high performance liquid chromatography-MS (HPLC-MS), capillary electrophoresis-MS, and nuclear magnetic resonance (NMR) spectrometry.

11. The method of claim 10, wherein the spectrometry is LC-MS/MS.

12. The method of claim 1, wherein the biological sample is selected from the group comprising urine, saliva, mucous, whole blood, and blood plasma.

13. The method of claim 12, wherein the biological sample is urine.

14. The method of claim 1, wherein the patient is negative for human immunodeficiency virus (HIV), positive for HIV, or at risk for HIV.

15. The method of claim 14, wherein the patient is negative for HIV, and said patient is at high risk for exposure to HIV.

16. The method of claim 1, wherein the metabolite has a concentration of about 0 ng/ml to about 10 ng/mL.

17. The method of claim 1, wherein the metabolite has a concentration of about 10 ng/ml to about 10,000 ng/mL.

18. The method of claim 12, wherein the metabolite has a concentration of about 10 ng/ml to about 1,000 ng/mL.

19. The method of claim 1, wherein the metabolite in step c) is contacted with a reagent to detect the metabolite.

20. The method of claim 19, wherein the reagent is an antibody.

21. The method of claim 20, wherein the antibody is a polyclonal antibody.

22. The method of claim 20, wherein the antibody is a monoclonal antibody.

23. The method of claim 1, wherein the prophylactic regimen is a pre-exposure prophylaxis (PrEP).

24. The method of claim 23, wherein the PrEP is for 28 days.

25. The method of claim 1, wherein the prophylactic region is a post exposure prophylaxis (PEP).

26. The method of claim 25, wherein the PEP is administered within 72 hours of a high risk exposure and continued for at least 28 days.

27. The method of claim 1, wherein in step d), recent adherence to the treatment or prophylactic regimen in the patient is determined.

28. The method of claim 27, wherein recent adherence is defined by a dose of NRTI within 48 hours.

29. The method of claim 27, wherein recent adherence is defined by a metabolite concentration of 1000 ng/mL or more.

30. The method of claim 27, wherein recent adherence to the prophylactic regimen in the patient identifies the patient as at little to no risk of contracting HIV.

31. The method of claim 1, wherein in step d), low adherence to the treatment or prophylactic regimen in the patient is determined.

32. The method of claim 31, wherein low adherence is defined by a dose of NRTI within 1 week.

33. The method of claim 31, wherein low adherence is defined by a metabolite concentration of 10 ng/mL to 999 ng/mL.

34. The method of claim 31, wherein low adherence to the prophylactic regimen in the patient identifies the patient as at risk of contracting HIV.

35. The method of claim 1, wherein in step d), non-adherence to the treatment or prophylactic regimen in the patient is determined.

36. The method of claim 35, wherein non-adherence is defined by a last dose of NRTI greater than 1 week.

37. The method of claim 35, wherein non-adherence is defined by a metabolite concentration of 10 ng/mL or less.

38. The method of claim 35, wherein non-adherence to the prophylactic regimen in the patient identifies the patient as at high risk of contracting HIV.

39. A diagnostic system for carrying out the method according to claim 1.

40. An immunoassay for carrying out the method according to claim 1.

41. The immunoassay of claim 40 which is a competitive assay.

42. A device for performing an assay to detect a metabolite in a fluid sample of a patient, wherein the patient is prescribed or administered an NRTI, comprising:
(a) a sample pad for contacting the fluid sample;
(b) a conjugated label pad, the conjugated label pad having a first reagent conjugated to a detectable label, a portion of the conjugated label pad and a portion of the sample pad forming a first interface;
(c) an assay comprising a membrane, a portion of the membrane and a portion of the conjugated label pad forming a second interface; and
(d) at least one second reagent bound to the membrane to form a test line, the first interface allowing fluid to flow from the sample pad to the conjugated label pad and contact the detectable label, the second interface allowing fluid to flow from the conjugated label pad to the membrane and to contact the at least one membrane-bound second reagent to form to a second reagent-first reagent complex, and cause the detectable label to form a detectable signal at the test line,
wherein the presence of a detectable signal indicates non-adherence to a treatment or prophylactic regimen in the patient, and wherein the absence of a detectable signal indicates adherence to a treatment or prophylactic regimen in the patient.

43. The device of claim 42, wherein the detectable signal is modulated to provide that the presence of a detectable signal indicates adherence to a treatment or prophylactic regimen in the patient.

44. The device of claim 42, which is a lateral flow assay.

45. The device of claim 44, which is a lateral flow immunoassay.

46. The device of claim 42, wherein the first reagent is an antibody conjugated to a detectable label.

47. The device of claim 42, wherein the first reagent is a conjugated derivative of the metabolite.

48. The device of claim 42, wherein the second reagent is a conjugated derivative of the metabolite.

49. The device of claim 42, wherein the second reagent is an antibody to the metabolite.

50. The device of claim 42, wherein the first reagent is an antibody conjugated to a detectable label and the second reagent is a conjugated derivative of the metabolite.

51. The device of claim 42, wherein the first reagent is a conjugated derivative of the metabolite and the second reagent is an antibody to the metabolite.

52. The device of claim 42, further comprising an absorbent pad downstream of the membrane.

53. The device of claim 42, wherein the membrane is nitrocellulose.

54. The device of claim 42, which is provided in a housing.

55. The device of claim 54, wherein the housing further comprises an opening for reading the detectable signal.

56. The device of claim 42, wherein the first reagent is an antibody specific for the metabolite.

57. The device of claim 56, wherein the antibody is a polyclonal antibody.

58. The device of claim 56, wherein antibody is a monoclonal antibody.

59. The device of claim 42, wherein the metabolite is TFV.

60. The device of claim 42, wherein the metabolite is a TFV derivative.

61. The device of claim 42, wherein the membrane further comprises a third reagent bound to the membrane downstream or upstream of the test line to form a control line.

62. The device of claim 61, wherein the third reagent binds to the first reagent to cause a detectable signal at the control line, wherein the presence of the detectable signal at the control line indicates proper performance of the lateral-flow assay.

63. The device of claim 42, which is a point of care test.

64. The device of claim 42 which is a cartridge.

65. The device of claim 42, wherein the fluid sample is urine.

66. The device of claim 42, wherein the prophylactic regimen is a PrEP to NRTI.

67. The device of claim 42, wherein the NRTI is selected from the group consisting of TDF, FTC, and TAF, or derivatives thereof or combinations thereof.

68. The device of claim 67, wherein the NRTI is TAF.

69. The device of claim 67, wherein the NRTI is TDF.

70. The device of claim 67, wherein the NRTI is FTC.

71. The device of claim 67, wherein the NRTI is a combination of TDF/FTC.

72. The device of claim 67, wherein the NRTI is a combination of TAF/FTC.

73. A kit, comprising:
(a) a sample collection receptacle for receiving a biological sample; and
(b) the device of claim 42 for assaying the biological sample;

74. The kit of claim 73 further comprising instructions for use.

75. The kit of claim 73 further comprising a hand held device.

76. The kit of claim 75, wherein the hand held device is a reader.

77. The kit of claim 76, wherein the reader is adapted to receive the device of claim 28.

78. The kit of claim 76, wherein the reader is a reflectance reader.

79. The kit of claim 73, wherein the sample is urine.
